(19) United States
(12) Patent Application Publication
(10) Pub. No.: US 2023/0287109 A1
(43) Pub. Date: Sep. 14, 2023

(54) VEGF and TIE2-Binding Fusion Protein and Uses Thereof

(71) Applicant: INGENIA Therapeutics Inc., Cambridge, MA (US)

(72) Inventors: Minkyung Choo, Cambridge, MA (US); Kwangsoo Kim, Ashland, MA (US); Sangyeul Han, Winchester, MA (US); Philip Durost, Auburn, MA (US)

(73) Assignee: INGENIA Therapeutics Inc., Cambridge, MA (US)

(21) Appl. No.: 18/153,965

(22) Filed: Jan. 12, 2023

Related U.S. Application Data
(60) Provisional application No. 63/299,177, filed on Jan. 13, 2022.
(60) Provisional application No. 63/310,359, filed on Feb. 15, 2022.
(60) Provisional application No. 63/335,805, filed on Apr. 28, 2022.

(51) Int. Cl. C07K 16/28; C07K 16/22; A61P 35/00; C12N 15/63

(52) U.S. Cl. CPC C07K 16/28; C07K 16/22; A61P 35/00; C12N 15/63; C07K 2317/565; C07K 2317/92; C07K 2317/31

(57) ABSTRACT

The present disclosure refers to a fusion protein having an antibody against Tie-2 or antigen-binding fragment thereof and a vascular endothelial growth factor (VEGF)-binding domain, methods for making the fusion protein, and pharmaceutical compositions and methods for preventing or treating angiogenic or vascular diseases or regulating angiogenesis, endothelial signaling, inflammation, and/or vascular leakage, which comprise the fusion protein.

### CROSS-REFERENCE TO RELATED APPLICATIONS

This application claims the benefit under 35 U.S.C. § 119(e) of U.S. Provisional Application No. 63/299,177, filed Jan. 13, 2022, U.S. Provisional Application No. 63/310,359, filed Feb. 15, 2022, and U.S. Provisional Application No. 63/335,805, filed Apr. 28, 2022. The entire disclosure of these earlier applications are hereby incorporated by reference in their entireties.

### SEQUENCE LISTING

The present application contains a Sequence Listing which has been submitted electronically in XML format. Said XML copy, created on Jan. 12, 2023, is named “2023-01-12_01262-0004-00US_ST26.xml” and is 48,487 bytes in size. The information in the electronic format of the sequence listing is incorporated herein by reference in its entirety.

### FIELD

The present disclosure relates to a fusion protein comprising an anti-Tie-2 antibody or an antigen-binding fragment thereof, and a vascular endothelial growth factor (VEGF)-binding domain wherein the fusion protein binds to Tie2 and VEGF simultaneously, a nucleic acid encoding the same, a vector containing the nucleic acid, a cell transformed with the vector, a method for preparing the fusion protein, methods and compositions for regulating angiogenesis, inflammation, and vascular leakage. In some embodiments, the present disclosure relates to methods and pharmaceutical compositions to prevent or treat angiogenic and vascular diseases.

### INTRODUCTION AND SUMMARY

Angiogenesis occurs dynamically by a variety of regulatory factors during the development, growth, maintenance, and homeostasis of an organism. Blood vessels newly formed in this process act as transport channels for various biomaterials such as nutrients, oxygen, and hormones in the surrounding cells. Functionally and structurally abnormal blood vessels are the direct or indirect cause for the initiation and progression of various diseases. Tumor blood vessels aggravate hypoxia due to their defective function and structure, resulting in tumor progression and metastasis to other tissues, in the poor delivery of anticancer drugs into the core of the tumor mass. Defective blood vessels are also found in other various diseases and conditions, in addition to cancer. Examples thereof include various ocular diseases (e.g., diabetic macular edema, wet age-related macular degeneration, diabetic retinopathy), viral infections, and acute inflammatory responses such as sepsis. Thus, if a therapeutic agent capable of normalizing pathologic blood vessels is available, it can be applied to the treatment of various patients with vascular abnormalities.

The angiopoietin family plays an important role in the formation and maintenance of blood vessels, and is comprised of four angiopoietins (Ang1, Ang2, Ang3, and Ang4). Angiopoietin-1 (Ang1) binds to the Tie2 receptor present on the surface of vascular endothelial cells to phosphorylate and activate Tie2 receptor, resulting in stabilization of blood vessels and suppression of vascular leakage. On the other hand, angiopoietin-2 (Ang2) binds to the Tie2 receptor, but acts as an antagonist to induce inactivation of the Tie2 receptor and inhibit binding by Ang1, resulting in destabilization of blood vessels and leakage of blood vessels, thereby tending to promote growth of new blood vessels. It was reported that the expression level of Ang2 is highly increased in the blood of cancer patients, ocular diseases, viral and bacterial infections and inflammatory diseases (Saharinen P et al., 2017, Nature Review Drug Discovery 16:635-661). However, Ang2 is also known to act as an agonist to induce activation of the Tie2 receptor in several processes, including lymphatic tube formation and maintenance, and thus it is believed that Ang2 performs various functions depending on the context.

Up to now, development and clinical testing of various Anti-Ang2 antibodies have been of interest to many biopharmaceutical companies (e.g., U.S. Pat. Nos. 7,658,924, and 8,987,420). These Ang2 antibodies are reported to inhibit the binding of Ang2 to Tie2 and the Ang2 neutralizing effect was reported to hinder the formation of new blood vessels. The anti-angiogenic and anti-cancer activities of these anti Ang2-antibodies have been demonstrated in many preclinical models, and diverse anti-Ang2 antibodies are being clinically tested in various cancer patients. However, their anti-cancer efficacy has been demonstrated to be insufficient. For example, Phase 3 clinical trials conducted by Amgen showed that the anti-cancer efficacy of the Ang2 antibody in ovarian cancer patients was insignificant (Marth C et al., 2017, Eur. J. Cancer, 70:111-121). In addition to cancer models, an Ang2 neutralizing antibody, Nesvacumab, was tested in ocular patients, however it failed to improve upon the efficacy of EYLEA® (anti-VEGF) in a clinical phase 2 study. See, e.g., “Regeneron provides update on EYLEA® (aflibercept) injection and nesvacumab (ang2 antibody) combination program” press release available at: investor.regeneron.com/news-releases/news-release-details/regeneron-provides-update-eylear-aflibercept-injection-and.

In contrast to the above-mentioned Ang2 neutralization approach, direct Tie2 activation has been also considered as an alternative approach to inhibit angiogenesis and suppress vascular permeability. Recombinant proteins, which bind directly to the Tie2 receptor and induce phosphorylation and activation of Tie2, have also been developed and tested in many preclinical cancer and ocular models. Examples thereof include cartilage oligomeric matrix protein (COMP)-Ang1 (Cho et al., 2004, PNAS 101(15): 5547-5552) and Vasculotide (David S et al., 2011, Am J Physiol Lung Cell Mol Physiol 300(6):L851-L862). Although these agents showed anti-angiogenic and anti-permeability activity, these tend to have very short half-lives and unstable physicochemical properties. In addition, a small molecule compound (AKB-9778) was developed as an inhibitor for a phosphatase, VE-PTP, which inactivates Tie2 by removing a phosphate group from phosphorylated Tie2 (Goel S et al., 2013, J Natl Cancer Inst 105(16):1188-1201). This compound indirectly increases Tie2 activity by inhibiting VE-PTP, although it has the disadvantage of activating other receptors as well. See, e.g., Frye M. et al., 2015, J Exp. Med., 212(13):2267-2287; Hayashi M, et al., 2013, Nature Communication, 4:1672; and Mellberg S. et al., 2009, FASEB J., 23(5):1490-1502). In addition, agonistic Tie2 antibodies have been developed. See, e.g., U.S. Pat. No. 6,365,154B1; and US Patent Publication No. 20170174789A1. These antibodies increased the survival of endothelial cells and inhibited the vascular leakage. Interestingly, herbal extracts were shown to activate Tie2 activity and claimed to be used as skin care cosmetics. See, e.g., Japanese Patent Application Nos. JP2011102273A, JP2018043949A, JP2015168656A).

Tie2 is a receptor protein that promotes the differentiation and stabilization of blood vessels and is highly expressed in blood vessels. If activated, the Tie2 receptor stabilizes blood vessels and it becomes possible to gather surrounding support cells. For example, activated Tie2 in cancer blood vessels normalizes the cancer vessels and reduces vascular leakage, eliminating the increased hypoxia within the tumor, supplying the sufficient oxygen by increasing blood flow into the tumor, and increasing the delivery of other anticancer drugs and the penetration of immune cells.

Vascular endothelial growth factor (VEGF) inhibitors are standard of care first-line treatment for diabetic macular edema (DME) and wet type age-related macular degeneration (wAMD), although moderate frequency of intraocular administration and the substantial inadequate responding population limit the efficacy of these treatments.

Accordingly, there is a need for improved methods for regulating angiogenesis, endothelial signaling, inflammation, and/or vascular leakage, and/or treating angiogenic diseases. For example, there is a need for treatments for angiogenic diseases, such as cancer, with improved efficacy, affinity, half-life, stability, pharmacodynamics, durability, and/or decreased frequency of treatment to reduce patient burden and compliance issues. The present disclosure aims to meet one or more of these needs, provide other benefits, or at least provide the public with a useful choice.

The present disclosure provides a fusion protein comprising an anti-Tie2 antibody or antigen-binding fragment thereof and a vascular endothelial growth factor (VEGF)-binding domain. In some embodiments, the fusion protein binds to Tie2 Ig3-FNIII (1-3) domain comprising SEQ ID NO: 2, 3, or 4 and to VEGF.

In one embodiment, the VEGF-binding domain comprises a VEGF receptor extracellular domain.

In one embodiment, the VEGF-binding domain is an antagonist VEGF-binding domain. Thus, in an embodiment, the VEGF-binding domain antagonizes (i.e., inhibits) the activity of VEGF in cells. The VEGF-binding domain achieves its antagonist effect by binding VEGF and thus preventing VEGF from exerting its activity.

In one embodiment, the VEGF-binding domain comprises an extracellular domain of two VEGF receptor isoforms, e.g., an extracellular domain of both VEGF receptor 1 and VEGF receptor 2 (e.g., an Ig2 domain of. VEGF receptor 1 and an Ig3 domain of VEGF receptor 2).

In one embodiment, the VEGF-binding domain comprises an anti-VEGF antibody or antibody binding fragment thereof. In a further embodiment, the anti-VEGF antibody or fragment thereof comprises a variable binding domain of bevacizumab, ranibizumab or HuMab G6-31 (US2007/0141065).

In some embodiments, the disclosure provides a fusion protein comprising a vascular endothelial growth factor (VEGF)-binding domain, the VEGF-binding domain comprising a vascular endothelial growth factor (VEGF)-A binding region of VEGF receptor 1 (VEGFR1) of SEQ ID NO: 13 and a vascular endothelial growth factor (VEGF)-A binding region of VEGF receptor 2 (VEGFR2) of SEQ ID NO: 14, and an anti-Tie2 antibody or antibody binding fragment thereof, wherein the fusion protein binds to Tie2 Ig3-FNIII (1-3) domain comprising SEQ ID NO: 2, 3, or 4, and VEGF.

In one embodiment, the anti-Tie2 antibody or antigen-binding fragment thereof is an agonist anti-Tie2 antibody or antigen-binding fragment thereof. Thus, in an embodiment, the anti-Tie2 antibody or antigen-binding fragment thereof binds Tie2 and provides, preserves or enhances Tie2 or Tie2 activity at the cell surface membrane.

In one embodiment, the VEGF-binding domain is linked to the C-terminus of the heavy chain (HC) of the anti-Tie2 antibody or antigen-binding fragment thereof.

In one embodiment, the fusion protein binds amino acids 633-644 (SEQ ID NO: 19) and/or amino acids 713-726 (SEQ ID NO: 20) of Tie2 of the amino acid sequence of SEQ ID NO:1. In one embodiment, the anti-Tie2 antibody or antibody binding fragment thereof binds amino acids 633-644 (SEQ ID NO: 19) and/or amino acids 713-726 (SEQ ID NO: 20) of Tie2 of the amino acid sequence of SEQ ID NO:1

In one embodiment, the anti-Tie2 antibody has an IgG1 isotype.

In one embodiment, the anti-Tie2 antibody or antigen-binding fragment thereof comprises a heavy chain variable region comprising heavy chain CDRs comprising amino acid sequences of SEQ ID NO:5-7 and a light chain variable region comprising a light chain CDRs comprising amino acid sequences of SEQ ID NO:8-10.

In one embodiment, the VEGF-binding domain comprises a sequence having at least 80%, 85%, 90%, 95%, 96%, 97%, 98%, or 99% identity to the amino acid sequence of SEQ ID NO: 15.

In one embodiment, the VEGF-binding domain comprises the amino acid sequence of SEQ ID NO: 15.

In one embodiment, the fusion protein comprises a linker between the VEGF-binding domain (e.g., VEGF receptor) and the anti-Tie2 antibody or antibody fragment thereof. In a further embodiment, the linker comprises between 5 and 50 amino acid residues, such as between 10 and 40 residues, such as between 15 and 30 residues, such as 20 residues.

In one embodiment, the fusion protein comprises a linker between the VEGF-binding domain (e.g., VEGF receptor) and the anti-Tie2 antibody or antibody fragment thereof, and wherein the linker comprises a sequence having at least 80%, 85%, 90%, 95%, 96%, 97%, 98%, or 99% identity to the amino acid sequence of SEQ ID NO: 16.

In one embodiment, the fusion protein comprises a linker between the VEGF-binding domain (e.g., VEGF receptor) and the anti-Tie2 antibody or antibody fragment thereof, and wherein the linker comprises the amino acid sequence of SEQ ID NO: 16.

In one embodiment, the fusion protein comprises a linker between the VEGF-binding domain (e.g., VEGF receptor) and the anti-Tie2 antibody or antibody fragment thereof, and wherein the linker comprises a sequence having at least 80%, 85%, 90%, 95%, 96%, 97%, 98%, or 99% identity to the amino acid sequence of SEQ ID NO: 25.

In one embodiment, the fusion protein comprises a linker between the VEGF-binding domain (e.g., VEGF receptor) and the anti-Tie2 antibody or antibody fragment thereof, and wherein the linker comprises the amino acid sequence of SEQ ID NO: 25.

In one embodiment, the fusion protein comprises a CH domain comprising a sequence having at least 80%, 85%, 90%, 95%, 96%, 97%, 98%, or 99% identity to the amino acid sequence of SEQ ID NO: 17.

In one embodiment, the fusion protein comprises a CH domain comprising the amino acid sequence of SEQ ID NO: 17.

In one embodiment, the fusion protein comprises one or more mutations in a constant region domain in the heavy chain to reduce or eliminate efficient interaction with Fc Receptors on other immune cells in the body. In one embodiment, the one or more mutations comprises altering L234, L235, G236, and G237 (EU numbering) to a LAGA mutation, a FEGG mutation, an AAGG mutation, an AAGA mutation, a LALA mutation, or a combination thereof. In one embodiment, the one or more mutations comprises a LALA mutation and mutations at K322 (e.g., K322A) and P331 (e.g., P331S) (EU numbering).

In one embodiment, the fusion protein comprises one or more mutations at L234, L235, H310, M252, 1253, S254, T256, H433, N434, and/or H435 (EU numbering). In one embodiment, the fusion protein comprises one or more mutations at L234A, L235A, and/or H310A. In one embodiment, the fusion protein comprises one or more mutations at M252 (e.g., M252Y), 1253 (e.g., I253A, I253M or I253V), S254 (e.g., S254T), T256 (e.g., T256D), H433 (e.g., H433K), N434 (e.g., N434F), and/or H435 (e.g., H435A, H435Q, or H435R).

In one embodiment, the fusion protein comprises, C-terminal to the heavy chain constant domain or domains of the anti-Tie2 antibody or antigen-binding fragment thereof and in N- to C-terminal order, a linker between the VEGF-binding domain and the anti-Tie2 antibody or antibody fragment thereof, such as wherein the linker comprises a sequence having between 5 and 50 residues, such as between 10 and 40 residues, such as between 15 and 30 residues, such as 20 residues, and the VEGF-binding domain comprises the amino acid sequence of SEQ ID NO: 15.

In one embodiment, the fusion protein comprises, C-terminal to the heavy chain constant domain or domains of the anti-Tie2 antibody or antigen-binding fragment thereof and in N- to C-terminal order, a linker between the VEGF-binding domain and the anti-Tie2 antibody or antibody fragment thereof, wherein the linker comprises amino acid sequence of SEQ ID NO: 16 or 25, and the VEGF-binding domain comprises the amino acid sequence of SEQ ID NO: 15.

In one embodiment, the fusion protein comprises a CH domain comprising the amino acid sequence of SEQ ID NO: 17, a linker comprising amino acid sequence of SEQ ID NO: 16 or 25 between the VEGF-binding domain and the anti-Tie2 antibody or antibody fragment thereof, and the VEGF-binding domain comprises the amino acid sequence of SEQ ID NO: 15.

In one embodiment, the fusion protein comprises a heavy chain variable region comprising the amino acid sequence of SEQ ID NO:18 and a light chain variable region comprising the amino acid sequence of SEQ ID NO:19.

In one embodiment, the fusion protein comprises a heavy chain comprising at least 80%, 85%, 90%, 95%, 96%, 97%, 98%, or 99% identity to the amino acid sequence of SEQ ID NO: 11.

In one embodiment, the fusion protein comprises a heavy chain comprising at least 80%, 85%, 90%, 95%, 96%, 97%, 98%, or 99% identity to the amino acid sequence of SEQ ID NO: 26.

In one embodiment, the fusion protein comprises a heavy chain comprising the amino acid sequence of SEQ ID NO: 11.

In one embodiment, the fusion protein comprises a heavy chain comprising the amino acid sequence of SEQ ID NO: 26.

In one embodiment, the fusion protein comprises a light chain comprising at least 80%, 85%, 90%, 95%, 96%, 97%, 98%, or 99% identity to the amino acid sequence of SEQ ID NO: 12.

In one embodiment, the fusion protein comprises a light chain comprising amino acid sequence of SEQ ID NO:12.

In one embodiment, the fusion protein comprises a heavy chain comprising amino acid sequence of SEQ ID NO:11, and a light chain comprising amino acid sequence of SEQ ID NO:12.

In one embodiment, the fusion protein comprises a heavy chain comprising amino acid sequence of SEQ ID NO: 26, and a light chain comprising amino acid sequence of SEQ ID NO:12.

In one embodiment, the fusion protein binds to Tie2 Ig3-FNIII (1-3) domain comprising SEQ ID NO: 2, 3, or 4 with an affinity K_{D} (M) of less than 3E⁻⁹M.

In one embodiment, the fusion protein is pegylated. PEGylation (or pegylation) refers to the covalent attachment or amalgamation of a chain comprising a plurality of polyethylene glycol (PEG, also referred to as macrogol) units (e.g., a PEG polymer). In one embodiment, the PEG has a molecular weight of about 40 kDa or about 20 kDa.

In one embodiment, the fusion protein is site-specifically pegylated. In one embodiment, the fusion protein is site-specifically pegylated on a cysteine residue. In one embodiment, the fusion protein further comprises the sequence of SEQ ID NO: 22 and is site-specifically pegylated on the cysteine residue of the sequence of SEQ ID NO: 22.

In one embodiment, the sequence of SEQ ID NO: 22 is present at the C-terminus of the heavy chain. In one embodiment, the heavy chain comprises the sequence of SEQ ID NO: 23. In one embodiment, the heavy chain comprises the sequence of SEQ ID NO: 24.

In one embodiment, the fusion protein further comprises one or more half-life extension modulators. In one embodiment, the one or more half-life extension modulators comprises a chemical, biopolymer, or peptide that increases the half-life of the fusion protein.

In one embodiment, there is provided a polypeptide comprising a chain monomer of the fusion protein of the invention.

In one embodiment, the polypeptide comprises the heavy chain monomer of the fusion protein of the invention. In a further embodiment, the polypeptide comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to the sequence of SEQ ID NO: 11 or 26. In one embodiment, the polypeptide consists of the sequence of SEQ ID NO: 11. In one embodiment, the polypeptide consists of the sequence of SEQ ID NO: 26.

In one embodiment, the polypeptide comprises the light chain monomer of the fusion protein of the invention. In a further embodiment, the polypeptide comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to SEQ ID NO: 12. In one embodiment, the polypeptide consists of the sequence of SEQ ID NO: 12.

As used herein, a “biopolymer” refers to polymers produced from natural sources, either chemically synthesized from a biological material or entirely biosynthesized by living organisms or microorganisms. In some embodiments, the biopolymer includes, but is not limited to, a polypeptide or protein (i.e., polymer of amino acids, e.g., collagen, actin, and fibrin), a polynucleotide (i.e., polymer of nucleic acids, e.g., DNA or RNA), polysaccharide (i.e., carbohydrates and glycosylated molecules, e.g., cellulose, starch, or alginate), natural rubbers (polymers of isoprene), subarin, lignin (complex polyphenolic polymers), cutin and cutan (complex polymers of long-chain fatty acids), melanin, metabolites, and other structural molecules.

In one embodiment, the one or more half-life extension modulators comprise: a biopolymer containing PEG (polyethylene-glycol), hyaluronic acid (HA), or phosphorylcholine; an albumin; an albumin-binding peptide; and/or an HA-binding protein fragment.

In another embodiment, the disclosure provides a nucleic acid encoding the fusion protein.

In another embodiment, the disclosure provides a nucleic acid encoding a polypeptide comprising a chain monomer of the fusion protein of the invention. In one embodiment, the disclosure provides a nucleic acid encoding the heavy chain of the fusion protein. In another embodiment, the disclosure provides a nucleic acid encoding the light chain of the fusion protein.

In one embodiment, the nucleic acid molecule comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to SEQ ID NO: 27 or 29. In one embodiment, the nucleic acid molecule consists of SEQ ID NO: 27 or 29.

In one embodiment. the nucleic acid molecule comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to SEQ ID NO: 28. In one embodiment, the nucleic acid molecule consists of SEQ ID NO: 28.

In one embodiment, the disclosure provides a set of one or more polynucleotides wherein each polynucleotide encodes at least one of the monomer chains of the fusion protein of the invention, such both chains (i.e., light and heavy chains) of said fusion protein are encoded.

In another embodiment, the disclosure provides an expression vector comprising the nucleic acid. In another embodiment, there is provided a vector which comprises nucleic acids encoding one heavy chain sequence and one light chain sequence of the fusion protein.

In one embodiment, there is provided a set of one or more vectors which collectively comprise the set of one or more polynucleotides of the invention, such that both chains (i.e., light and heavy chains) of said fusion protein are encoded in the set of vectors.

In one embodiment, the vector is an animal virus, such as a virus selected from reverse transcriptase virus (including lentivirus), adenovirus, adeno-associated virus, herpes virus, chicken pox virus, baculovirus, papilloma virus, anellovirus, and papova virus.

In one embodiment, the expression vector further comprises a human cytomegalovirus IE1 (CMV-IE1) promoter/enhancer.

In another embodiment, the disclosure provides a cell transformed with the expression vector.

In another embodiment, the disclosure provides a method of manufacturing a fusion protein which binds Tie2 and VEGF, comprising the steps of: culturing a cell transformed with the expression vector; and recovering a fusion protein from the cultured cell. In another embodiment, the disclosure provides a process for the production of a fusion protein of the invention by expression from a vector or set of vectors.

In another embodiment, the disclosure provides a method for preventing or treating an angiogenic disease, comprising administering and effective amount of a fusion protein to a subject in need thereof.

In another embodiment, the disclosure provides for use of the fusion protein for the manufacture of a medicament for treating an angiogenic disease in a subject in need thereof.

In another embodiment, the disclosure provides the fusion protein for use in treating an angiogenic or vascular disease in a subject in need thereof.

In some embodiments, the angiogenic or vascular disease is cancer, metastasis, diabetic retinopathy, retinopathy of prematurity, diabetic macular edema, corneal graft rejection, macular degeneration, glaucoma such as neovascular glaucoma, systemic erythrosis, proliferative retinopathy, psoriasis, hemophilic arthritis, allied sclerosis, capillary formation of atherosclerotic plaques, keloid, wound granulation, vascular adhesion, rheumatoid arthritis, osteoarthritis, autoimmune diseases, Crohn's disease, restenosis, atherosclerosis, intestinal adhesions, cat scratch disease, ulcer, liver cirrhosis, nephritis, diabetic nephropathy, diabetes mellitus, an inflammatory disease, or a neurodegenerative disease.

In some embodiments, the cancer is esophageal cancer, stomach cancer, large intestine cancer, rectal cancer, oral cancer, pharyngeal cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, or multiple myeloid blood cancer.

In some embodiments, the disclosure provides a method for regulating angiogenesis, endothelial signaling, inflammation, and/or vascular leakage, comprising administering an effective amount of a fusion protein to a subject in need thereof.

In some embodiments, the inflammation is from sepsis, acute respiratory distress syndromes, and/or virus-infectious diseases.

In some embodiments, the subject is human. In some embodiments, the subject is a companion animal, such as a mammalian companion animal. In some embodiments, the mammalian companion animal is a dog, cat, rabbit, ferret, horse, mule, donkey, or hamster or other domesticated pet.

In some embodiments, the disclosure provides a pharmaceutical composition comprising the fusion protein. In some embodiments, the pharmaceutical composition further comprises a pharmaceutically acceptable carrier, diluent, or excipient.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims. Section headings are provided solely for the convenience of the reader and do not limit the disclosure.

The accompanying drawings, which are incorporated in and constitute a part of this specification, illustrate certain embodiments and together with the description, serve to explain the principles described herein.

### BRIEF DESCRIPTION OF THE DRAWINGS

FIGS. 1A-1C provide sensorgrams for binding of IGT-427 to the human (FIG. 1A), rabbit (FIG. 1B), and mouse (FIG. 1C) Tie2-Fc fusion protein.

FIGS. 2A-2C show inhibition of VEGFR2 phosphorylation and AKT activation by IGT-427. Human umbilical vein endothelial cells (HUVECs) were serum-starved for 4 hours and incubated with IGT-427 (FIG. 2A) or aflibercept (FIG. 2B) at the indicated concentrations for 30 min, and then treated with recombinant human VEGF for 2 min Cell lysates were subjected to SDS-PAGE/Western blotting and blots were probed with anti-phospho-VEGFR2 (Tyr1175), anti-VEGFR2, anti-phospho-Akt (S473), or total Akt antibodies. VEGF reporter assay shows comparable inhibitory effects of IGT-427, faricimab, and aflibercept (FIG. 2C).

FIG. 3 shows stronger and more persistent levels of phospho-Tie2 signaling by IGT-427, when compared with Angiopoietin-1. Chinese Hamster Ovary (CHO) cells overexpressing human Tie2 was serum starved for 4 hours and then treated with 10 nM of IGT-427 or Angiopoietin-1 for indicated durations. The lysates were subject to phospho-Tie2 and total Tie2 ELISA assays.

FIG. 4 shows dose-dependent activation of Akt signaling by IGT-427 in HUVECs.

FIG. 5 shows that IGT-427 overcomes and bypasses angiopoietin-2's effect on Tie2 signaling in HUVECs, leading to further activation of Akt signaling in the presence of pre-treated Ang2.

FIG. 6 shows that IGT-427 binds to Tie2 in complex with Ang2 and VEGF simultaneously. Ang2 was captured in CM5 chip and human Tie2, IGT-427 and human VEGF were subsequently injected in surface plasmon resonance (SPR) (Biocore™) analysis.

FIG. 7 shows inhibition of TNF-alpha induced-apoptosis by IGT-427. HUVECs were pre-treated with IGT-427 or EYLEA® for 1 hour and then treated with TNF (50 ng/ml) for 24 hr. Apoptotic cells was stained by APO-BrdU™ TUNEL Assay Kit (Thermo Fisher, A23210), and determined by Attune (Thermo Fisher). Values are mean±SE. ***p<0.001, ****p<0.0001 by one-way ANOVA.

FIG. 8 shows the cell surface levels of Tie2 on the Chinese Hamster Ovary cells overexpressing human Tie2 (CHO-Tie2) treated with various agents at various time points.

FIG. 9 shows that IGT-427 blocks Tie2 cleavage by MMP14. Recombinant human MMP14, human Tie2-ECD-human IgG Fc fusion proteins, or IGT-427 was mixed and incubated as indicated.

FIG. 10 shows that IGT-427 inhibits sTie2 generation in basal or TNF-alpha treated HUVECs. The levels of sTie2 were measured by Tie2 ELISA assay.

FIG. 11 shows that IGT-427 recovers compromised endothelial barrier integrity by VEGF treatment. TEER (trans-endothelial electrical resistance) was assessed in HUVECs.

FIG. 12 shows suppression of CNV (Choroidal Neo-Vascularization) by intravitreally injected IGT-427 or EYLEA® in laser-induced CNV model. The intravitreal administration of antibodies (50 μl injection volume/eye, EYLEA® (800 μg), IGT-427 (885 μg), control IgG (716 μg)) was performed at 0 day after laser photocoagulation. The molar ratio of EYLEA®, IGT-427 & control IgG is 1:0.65:0.68. Fluorescence intensity in the leaky areas around CNV was measured in FA images taken at 14 days after laser photocoagulation. For each group, 4-7 rabbits were tested where 6 laser-induced CNV lesions were tested for each rabbit. CNV lesions (n=24 −42) were imaged for each group. Values are mean±SEM. *p<0.05, **p<0.001 by one-way ANOVA.

FIG. 13 shows schematic drawings of IGT-427 variants for PEGylation. All constructs have a common light chain with five different heavy chains that generate two labile cysteines per antibody.

FIG. 14 shows SDS-PAGE data of optimized PEGylation conditions for the five IGT-427 variants. PRO593, PRO594, and PRO595 undergo complete conversion with no unmodified antibody in the reaction mixture. The reaction mixtures of PRO592 and PRO596 have some unmodified antibody at the end of the reaction.

FIG. 15 shows SPR binding of IGT-427 and PEGylation variants to Tie2 and VEGF surfaces. There are slight differences in binding signal to either antigen, but relative to unmodified IGT-427, the PEGylation variants bind both antigens similarly.

FIG. 16 shows SEC-HPLC chromatograms of the five ocular PK study test articles. All constructs were greater than 90% pure, with the exception of the 20 kDa PEGylated species, which contained 15% high molecular weight species.

FIG. 17 shows non-reduced SDS-PAGE data of ocular PK study test articles. Lane 1: EYLEA®; Lane 2: Faricimab; Lane 3: IGT-427; Lane 4: (2×20 kDa linear PEG)-IGT-427; Lane 5: (2×40 kDa branched PEG)-IGT-427.

FIGS. 18A-18C show SPR binding data of ocular PK test articles to rabbit VEGF (FIG. 18A), Tie2 (FIG. 18B), and Ang2 (FIG. 18C). A summary of the measured binding constants is tabulated.

FIG. 19 shows summary of total drug measurements from New Zealand white rabbit vitreous. Measured drug levels from ELISA quantification plotted over time. Each data point represents a duplicate measurements from a single rabbit eye. Exponential fits to each dataset are shown in red with fitted parameters and extracted ocular half-life displayed below each curve. For PEGylated IGT-427 variants, 14-day measurements are included in the plots but excluded from the fitting and extracted half-life analysis. A summary of the observed half-lives for each test article is shown in the bottom right.

### DESCRIPTION OF THE SEQUENCES

Table 1 provides a listing of certain sequences referenced herein.

Description of the Sequences

| | | | SEQ |
|---|---|---|---|
| | | | ID |
| Description | Sequences | | NO. |
| Human Tie2 | MDSLASLVLCGVSLLLSGTVEGAMDLILINSLPLVSDAETSLTCIASGWR | 50 | 1 |
| Full-Length | PHEPITIGRDFEALMNQHQDPLEVTQDVTREWAKKVVWKREKASKINGAY | 100 | |
| | FCEGRVRGEAIRIRTMKMRQQASFLPATLTMTVDKGDNVNISFKKVLIKE | 150 | |
| | EDAVIYKNGSFIHSVPRHEVPDILEVHLPHAQPQDAGVYSARYIGGNLFT | 200 | |
| | SAFTRLIVRRCEAQKWGPECNHLCTACMNNGVCHEDTGECICPPGFMGRT | 250 | |
| | CEKACELHTFGRTCKERCSGQEGCKSYVFCLPDPYGCSCATGWKGLQCNE | 300 | |
| | ACHPGFYGPDCKLRCSCNNGEMCDRFQGCLCSPGWQGLQCEREGIQRMTP | 350 | |
| | KIVDLPDHIEVNSGKFNPICKASGWPLPTNEEMTLVKPDGTVLHPKDFNH | 400 | |
| | TDHFSVAIFTIHRILPPDSGVWVCSVNTVAGMVEKPFNISVKVLPKPLNA | 450 | |
| | PNVIDTGHNFAVINISSEPYFGDGPIKSKKLLYKPVNHYEAWQHIQVTNE | 500 | |
| | IVTLNYLEPRTEYELCVQLVRRGEGGEGHPGPVRRFTTASIGLPPPRGLN | 550 | |
| | LLPKSQTTLNLTWQPIFPSSEDDFYVEVERRSVQKSDQQNIKVPGNLTSV | 600 | |
| | LLNNLHPREQYVVRARVNTKAQGEWSEDLTAWTLSDILPPQPENIKISNI | 650 | |
| | THSSAVISWTILDGYSISSITIRYKVQGKNEDQHVDVKIKNATITQYQLK | 700 | |
| | GLEPETAYQVDIFAENNIGSSNPAFSHELVTLPESQAPADLGGGKMLLIA | 750 | |
| | ILGSAGMTCLTVLLAFLIILQLKRANVQRRMAQAFQNVREEPAVQFNSGT | 800 | |
| | LALNRKVKNNPDPTIYPVLDWNDIKFQDVIGEGNFGQVLKARIKKDGLRM | 850 | |
| | DAAIKRMKEYASKDDHRDFAGELEVLCKLGHHPNIINLLGACEHRGYLYL | 900 | |
| | AIEYAPHGNLLDFLRKSRVLETDPAFAIANSTASTLSSQQLLHFAADVAR | 950 | |
| | GMDYLSQKQFIHRDLAARNILVGENYVAKIADFGLSRGQEVYVKKTMGRL | 1000 | |
| | PVRWMAIESLNYSVYTTNSDVWSYGVLLWEIVSLGGTPYCGMTCAELYEK | 1050 | |
| | LPQGYRLEKPLNCDDEVYDLMRQCWREKPYERPSFAQILVSLNRMLEERK | 1100 | |
| | TYVNTTLYEKFTYAGIDCSAEEAA | 1124 | |
| Human Tie2 | TPKIVDLPDHIEVNSGKFNPICKASGWPLPTNEEMTLVKPDGTVLHPKDF | 50 | 2 |
| Ig3-FNIII | NHTDHFSVAIFTIHRILPPDSGVWVCSVNTVAGMVEKPFNISVKVLPKPL | 100 | |
| (1-3) | NAPNVIDTGHNFAVINISSEPYFGDGPIKSKKLLYKPVNHYEAWQHIQVT | 150 | |
| | NEIVTLNYLEPRTEYELCVQLVRRGEGGEGHPGPVRRFTTASIGLPPPRG | 200 | |
| | LNLLPKSQTTLNLTWQPIFPSSEDDFYVEVERRSVQKSDQQNIKVPGNLT | 250 | |
| | SVLLNNLHPREQYVVRARVNTKAQGEWSEDLTAWTLSDILPPQPENIKIS | 300 | |
| | NITHSSAVISWTILDGYSISSITIRYKVQGKNEDQHVDVKIKNATITQYQ | 350 | |
| | LKGLEPETAYQVDIFAENNIGSSNPAFSHELVTLPESQAP | 390 | |
| Rabbit Tie2 | TPKIEDLPDHIEVNTGKFNPICKASGWPLPANEEMTLVKPDGTVLHPKDFNHTENFSVAI | 60 | 3 |
| Ig3-FNIII | FTIHRILPLDSGVWVCSVNTVAGMVEKPFNISVKVLPKPLNAPNVIDTGHNFAVINISSE | 120 | |
| (1-3) | PYFGDGPIKSKKLLYKPVNDYEAWRHIQVTNEIVTLNYLEPRTEYELCVQLIRRGEGGEG | 180 | |
| | HPGPVRRFTTASIGLPPPQGLILLPKSQTTLNLTWQPIFPSSEDDFYVEVERRSVQIKSD | 240 | |
| | QQNIKVPGNLTSVLLNNLHPREQYVVRARVNTKAQGEWSEDLTAWTLSDIVPPQPENIKI | 300 | |
| | SNITDSSAVISWTILDGYSISSIIIRYKVQGKNEDQHIDVKIKNATITQYQLKGLEPETA | 360 | |
| | YQVDMFAENNIGSSNPAFSHELMTLPESQAP | 391 | |
| Mouse Tie2 | TPQIEDLPDHIEVNSGKFNPICKASGWPLPTSEEMTLVKPDGTVLQPNDFNYTDRFSVAI | 60 | 4 |
| Ig3-FNIII | FTVNRVLPPDSGVWVCSVNTVAGMVEKPFNISVKVLPEPLHAPNVIDTGHNFAIINISSE | 120 | |
| (1-3) | PYFGDGPIKSKKLFYKPVNQAWKYIEVTNEIFTLNYLEPRTDYELCVQLARPGEGGEGHP | 180 | |
| | GPVRRFTTASIGLPPPRGLSLLPKSQTALNLTWQPIFTNSEDEFYVEVERRSLQTTSDQQ | 240 | |
| | NIKVPGNLTSVLLSNLVPREQYTVRARVNTKAQGEWSEELRAWTLSDILPPQPENIKISN | 300 | |
| | ITDSTAMVSWTIVDGYSISSIIIRYKVQGKNEDQHIDVKIKNATVTQYQLKGLEPETTYH | 360 | |
| | VDIFAENNIGSSNPAFSHELRTLPHSPAS | 389 | |
| Antibody | SYWMN | | 5 |
| 3H7 | | | |
| CDRH1- | | | |
| KABAT | | | |
| Antibody | MIHPSDSETRLNQKFMD | | 6 |
| 3H7 | | | |
| CDRH2- | | | |
| KABAT | | | |
| Antibody | GLYGNS | | 7 |
| 3H7 | | | |
| CDRH3- | | | |
| KABAT | | | |
| Antibody | RASQDIGISLN | | 8 |
| 3H7 | | | |
| CDRL1- | | | |
| KABAT | | | |
| Antibody | ATSSLDS | | 9 |
| 3H7 | | | |
| CDRL2- | | | |
| KABAT | | | |
| Antibody | LQYASSPYT | | 10 |
| 3H7 | | | |
| CDRL3- | | | |
| KABAT | | | |
| IGT-427 HC | MNFGLRLIFLVLTLKGVQCEVQLVQSGAEVKKPGASVKVSCKASGYSFTSYWMNWVRQAP | 60 | 11 |
| with (G₄S)₄ | GQGLEWMGMIHPSDSETRLNQKFMDRVTMTRDTSTSTVYMELSSLRSEDTAVYYCARGLY | 120 | |
| linker | GNSWGQGTLVTVSSASTKGPSVFPLAPSSKSTSGGTAALGCLVKDYFPEPVTVSWNSGAL | 180 | |
| | TSGVHTFPAVLQSSGLYSLSSVVTVPSSSLGTQTYICNVNHKPSNTKVDKKVEPKSCDKT | 240 | |
| | HTCPPCPAPEAAGGPSVFLFPPKPKDTLMISRTPEVTCVVVDVSHEDPEVKFNWYVDGVE | 300 | |
| | VHNAKTKPREEQYNSTYRVVSVLTVLAQDWLNGKEYKCKVSNKALPAPIEKTISKAKGQP | 360 | |
| | REPQVYTLPPSRDELTKNQVSLTCLVKGFYPSDIAVEWESNGQPENNYKTTPPVLDSDGS | 420 | |
| | FFLYSKLTVDKSRWQQGNVFSCSVMHEALHNHYTQKSLSLSPGKGGGGSGGGGSGGGGSG | 480 | |
| | GGGSSDTGRPFVEMYSEIPEIIHMTEGRELVIPCRVTSPNITVTLKKFPLDTLIPDGKRI | 540 | |
| | IWDSRKGFIISNATYKEIGLLTCEATVNGHLYKTNYLTHRQTNTIIDVVLSPSHGIELSV | 600 | |
| | GEKLVLNCTARTELNVGIDFNWEYPSSKHQHKKLVNRDLKTQSGSEMKKFLSTLTIDGVT | 660 | |
| | RSDQGLYTCAASSGLMTKKNSTFVRVHEK | 689 | |
| IGT-427 LC | MNFGLRLIFLVLTLKGVQCDIQMTQSPSSLSASVGDRVTITCRASQDIGISLNWYQQEPG | 60 | 12 |
| | KAIKRLIYATSSLDSGVPKRFSGSRSGTEYTLTISSLESEDFADYYCLQYASSPYTFGGG | 120 | |
| | TKLEIKRTVAAPSVFIFPPSDEQLKSGTASVVCLLNNFYPREAKVQWKVDNALQSGNSQE | 180 | |
| | SVTEQDSKDSTYSLSSTLTLSKADYEKHKVYACEVTHQGLSSPVTKSFNRGEC | 233 | |
| VEGFR1 | SDTGRPFVEMYSEIPEIIHMTEGRELVIPCRVTSPNITVTLKKFPLDTLIPDGKRIIWDS | | 13 |
| | RKGFIISNATYKEIGLLTCEATVNGHLYKTNYLTHRQTNTIIDV | | |
| VEGFR2 | VLSPSHGIELSVGEKLVLNCTARTELNVGIDFNWEYPSSKHQHKKLVNRDLKTQSGSEMK | | 14 |
| | KFLSTLTIDGVTRSDQGLYTCAASSGLMTKKNSTFVRVHEK | | |
| Recombinant | APMAEGGGQNHHEVVKFMDVYQRSYCHPIETLVDIFQEYPDEIEYIFKPSCVPLMRCGGC | | 15 |
| Human | CNDEGLECVPTEESNITMQIMRIKPHQGQHIGEMSFLQHNKCECRPKKDRARQENPCGPC | | |
| VEGF 165 | SERRKHLFVQDPQTCKCSCKNTDSRCKARQLELNERTCRCDKPRR | | |
| Protein, | | | |
| VEGFR | | | |
| Binding | | | |
| Domain | | | |
| (G₄S)₄ Linker | GGGGSGGGGSGGGGSGGGGS | | 16 |
| IGT-427 CH | ASTKGPSVFPLAPSSKSTSGGTAALGCLVKDYFPEPVTVSWNSGALTSGVHTFPAVLQSS | | 17 |
| Domain | GLYSLSSVVTVPSSSLGTQTYICNVNHKPSNTKVDKKVEPKSCDKTHTCPPCPAPEAAGG | | |
| | PSVFLFPPKPKDTLMISRTPEVTCVVVDVSHEDPEVKFNWYVDGVEVHNAKTKPREEQYN | | |
| | STYRVVSVLTVLAQDWLNGKEYKCKVSNKALPAPIEKTISKAKGQPREPQVYTLPPSRDE | | |
| | LTKNQVSLTCLVKGFYPSDIAVEWESNGQPENNYKTTPPVLDSDGSFFLYSKLTVDKSRW | | |
| | QQGNVFSCSVMHEALHNHYTQKSLSLSPGK | | |
| IGT-427 VH | EVQLVQSGAEVKKPGASVKVSCKASGYSFTSYWMNWVRQAPGQGLEWMGMIHPSDSETRL | | 18 |
| (HC Variable | NQKFMDRVTMTRDTSTSTVYMELSSLRSEDTAVYYCARGLYGNSWGQGTLVTVS | | |
| Region) | | | |
| IGT-427 VL | DIQMTQSPSSLSASVGDRVTITCRASQDIGISLNWYQQEPGKAIKRLIYATSSLDSGVPK | | 19 |
| (LC Variable | RFSGSRSGTEYTLTISSLESEDFADYYCLQYASSPYTFGGGTKLEI | | |
| Region) | | | |
| Binding | TLSDILPPQPEN | | 20 |
| domain | | | |
| (amino acids | | | |
| 633-644 of | | | |
| Tie2 of SEQ | | | |
| ID NO: 1) | | | |
| Binding | FAENNIGSSNPAFS | | 21 |
| domain | | | |
| (amino acids | | | |
| 713-726 of | | | |
| Tie2 of SEQ | | | |
| ID NO: 1) | | | |
| C-terminal | GGSSKCA | | 22 |
| addition | | | |
| IGT-427 HC | MNFGLRLIFLVLTLKGVQCEVQLVQSGAEVKKPGASVKVSCKASGYSFTSYWMNWVRQAP | 60 | 23 |
| with C- | GQGLEWMGMIHPSDSETRLNQKFMDRVTMTRDTSTSTVYMELSSLRSEDTAVYYCARGLY | 120 | |
| terminal | GNSWGQGTLVTVSSASTKGPSVFPLAPSSKSTSGGTAALGCLVKDYFPEPVTVSWNSGAL | 180 | |
| addition | TSGVHTFPAVLQSSGLYSLSSVVTVPSSSLGTQTYICNVNHKPSNTKVDKKVEPKSCDKT | 240 | |
| | HTCPPCPAPEAAGGPSVFLFPPKPKDTLMISRTPEVTCVVVDVSHEDPEVKFNWYVDGVE | 300 | |
| | VHNAKTKPREEQYNSTYRVVSVLTVLAQDWLNGKEYKCKVSNKALPAPIEKTISKAKGQP | 360 | |
| | REPQVYTLPPSRDELTKNQVSLTCLVKGFYPSDIAVEWESNGQPENNYKTTPPVLDSDGS | 420 | |
| | FFLYSKLTVDKSRWQQGNVFSCSVMHEALHNHYTQKSLSLSPGKGGGGSGGGGSGGGGSG | 480 | |
| | GGGSSDTGRPFVEMYSEIPEIIHMTEGRELVIPCRVTSPNITVTLKKFPLDTLIPDGKRI | 540 | |
| | IWDSRKGFIISNATYKEIGLLTCEATVNGHLYKTNYLTHRQTNTIIDVVLSPSHGIELSV | 600 | |
| | GEKLVLNCTARTELNVGIDFNWEYPSSKHQHKKLVNRDLKTQSGSEMKKFLSTLTIDGVT | 660 | |
| | RSDQGLYTCAASSGLMTKKNSTFVRVHEKGGSSKCA | 696 | |
| VEGFR2 | VLSPSHGIELSVGEKLVLNCTARTELNVGIDFNWEYPSSKHQHKKLVNRDLKTQSGSEMK | | 24 |
| with C- | KFLSTLTIDGVTRSDQGLYTCAASSGLMTKKNSTFVRVHEKGGSSKCA | | |
| terminal | | | |
| addition | | | |
| sequence | | | |
| from IGT- | | | |
| 427 HC | | | |
| IgG₁ Linker | GGGGSGGGGSEPKSSDKTHT | | 25 |
| IGT-427 HC | MGWSCIILFLVATATGVHSEVQLVQSGAEVKKPGASVKVSCKASGYSFTSYWMNWVRQAP | | 26 |
| with IgG₁ | GQGLEWMGMIHPSDSETRLNQKFMDRVTMTRDTSTSTVYMELSSLRSEDTAVYYCARGLY | | |
| linker | GNSWGQGTLVTVSSASTKGPSVFPLAPSSKSTSGGTAALGCLVKDYFPEPVTVSWNSGAL | | |
| | TSGVHTFPAVLQSSGLYSLSSVVTVPSSSLGTQTYICNVNHKPSNTKVDKKVEPKSCDKT | | |
| | HTCPPCPAPEAAGGPSVFLFPPKPKDTLMISRTPEVTCVVVDVSHEDPEVKFNWYVDGVE | | |
| | VHNAKTKPREEQYNSTYRVVSVLTVLAQDWLNGKEYKCKVSNKALPAPIEKTISKAKGQP | | |
| | REPQVYTLPPSRDELTKNQVSLTCLVKGFYPSDIAVEWESNGQPENNYKTTPPVLDSDGS | | |
| | FFLYSKLTVDKSRWQQGNVFSCSVMHEALHNHYTQKSLSLSPGKGGGGSGGGGSEPKSSD | | |
| | KTHTSDTGRPFVEMYSEIPEIIHMTEGRELVIPCRVTSPNITVTLKKFPLDTLIPDGKRI | | |
| | IWDSRKGFIISNATYKEIGLLTCEATVNGHLYKTNYLTHRQTNTIIDVVLSPSHGIELSV | | |
| | GEKLVLNCTARTELNVGIDFNWEYPSSKHQHKKLVNRDLKTQSGSEMKKFLSTLTIDGVT | | |
| | RSDQGLYTCAASSGLMTKKNSTFVRVHEK | | |
| Nucleotide | ATGGGATGGTCATGTATCATCCTTTTTCTGGTAGCAACTGCAACTGGAGTACATAGCGAG | | 27 |
| sequence of | GTGCAGCTGGTGCAATCAGGCGCTGAGGTCAAGAAACCCGGGGCTTCAGTCAAGGTGAGC | | |
| IGT-427 HC | TGTAAGGCTTCTGGCTACTCTTTCACCTCCTACTGGATGAATTGGGTCAGACAGGCCCCT | | |
| with (G₄S)₄ | GGGCAAGGACTGGAGTGGATGGGCATGATCCACCCAAGCGACAGTGAAACACGCCTGAAC | | |
| linker | CAGAAGTTTATGGACCGTGTCACCATGACAAGAGACACCAGCACAAGCACTGTGTATATG | | |
| | GAGCTCTCTTCCCTGCGCTCCGAAGATACCGCCGTGTACTACTGCGCCCGGGGACTCTAT | | |
| | GGAAACTCCTGGGGCCAGGGCACCCTAGTGACAGTGTCCTCTGCTAGCACCAAGGGCCCA | | |
| | TCGGTCTTCCCCCTGGCACCCTCCTCCAAGAGCACCTCTGGGGGCACAGCGGCCCTGGGC | | |
| | TGCCTGGTCAAGGACTACTTCCCCGAACCGGTGACGGTGTCGTGGAACTCAGGCGCCCTG | | |
| | ACCAGCGGCGTGCACACCTTCCCGGCCGTCCTACAGTCCTCAGGACTCTACTCCCTCAGC | | |
| | AGCGTGGTGACCGTGCCCTCCAGCAGCTTGGGCACCCAGACCTACATCTGCAACGTGAAT | | |
| | CACAAGCCCAGCAACACCAAGGTGGACAAGAAGGTTGAGCCCAAATCTTGTGACAAAACT | | |
| | CACACATGCCCACCGTGCCCAGCACCTGAAGCCGCTGGGGGACCGTCAGTCTTCCTCTTC | | |
| | CCCCCAAAACCCAAGGACACCCTCATGATCTCCCGGACCCCTGAGGTCACATGCGTGGTG | | |
| | GTGGACGTGAGCCACGAAGACCCTGAGGTCAAGTTCAACTGGTACGTGGACGGCGTGGAG | | |
| | GTGCATAATGCCAAGACAAAGCCGCGGGAGGAGCAGTACAACAGCACGTACCGTGTGGTC | | |
| | AGCGTCCTCACCGTCCTGGCCCAGGACTGGCTGAATGGCAAGGAGTACAAGTGCAAGGTC | | |
| | TCCAACAAAGCCCTCCCAGCCCCCATCGAGAAAACCATCTCCAAAGCCAAAGGGCAGCCC | | |
| | CGAGAACCACAGGTGTACACCCTGCCCCCATCCCGGGACGAGCTGACCAAGAACCAGGTC | | |
| | AGCCTGACCTGCCTGGTCAAAGGCTTCTATCCCAGCGACATCGCCGTGGAGTGGGAGAGC | | |
| | AATGGGCAGCCGGAGAACAACTACAAGACCACGCCTCCCGTGCTGGACTCCGACGGCTCC | | |
| | TTCTTCCTCTACAGCAAGCTCACCGTGGACAAGAGCAGGTGGCAGCAGGGGAACGTCTTC | | |
| | TCATGCTCCGTGATGCATGAGGCTCTGCACAACCACTACACGCAGAAGAGCCTCTCCCTG | | |
| | TCTCCGGGCAAAGGTGGCGGAGGCTCTGGAGGCGGCGGATCCGGCGGAGGAGGCTCCGGA | | |
| | GGAGGTGGCAGCAGTGATACGGGAAGACCTTTCGTGGAGATGTACTCTGAGATCCCCGAA | | |
| | ATTATTCACATGACCGAGGGCCGGGAGCTGGTCATCCCTTGCCGAGTGACAAGCCCAAAC | | |
| | ATCACTGTTACTCTCAAGAAGTTTCCTCTCGACACCCTGATACCGGATGGCAAGAGAATC | | |
| | ATTTGGGATTCCCGCAAGGGCTTCATCATCTCTAACGCTACATATAAGGAGATCGGACTG | | |
| | CTGACATGCGAAGCCACCGTGAATGGCCACCTCTACAAAACCAACTACCTGACCCACCGC | | |
| | CAAACAAATACCATCATTGACGTGGTTCTATCCCCAAGCCATGGGATTGAACTGAGTGTC | | |
| | GGAGAAAAGTTAGTGCTGAACTGCACCGCACGCACGGAACTGAACGTCGGCATCGACTTT | | |
| | AATTGGGAGTACCCCTCTAGCAAGCACCAGCACAAAAAGCTGGTTAATCGAGACCTGAAG | | |
| | ACCCAGAGCGGTTCTGAAATGAAGAAATTCCTGTCCACCCTGACTATTGACGGGGTAACC | | |
| | AGGTCGGATCAAGGGCTGTATACTTGCGCGGCGTCTAGTGGCTTGATGACAAAGAAGAAC | | |
| | AGCACCTTCGTTAGAGTGCATGAGAAA | | |
| Nucleotide | ATGGAGACAGACACACTCCTGCTATGGGTACTGCTGCTCTGGGTTCCAGGCTCCACCGGC | | 28 |
| sequence of | GACATTCAGATGACCCAATCGCCGTCTTCCCTCTCTGCTAGCGTCGGCGACCGAGTTACC | | |
| IGT-427 LC | ATCACTTGTCGAGCTAGCCAGGACATCGGTATCTCACTGAATTGGTACCAGCAAGAGCCA | | |
| | GGAAAAGCCATCAAGAGGCTGATATATGCCACAAGTTCACTGGATAGCGGAGTGCCCAAA | | |
| | AGGTTTAGTGGCAGTAGAAGCGGCACAGAGTATACCCTGACTATCTCAAGCCTGGAGTCC | | |
| | GAGGACTTTGCTGACTACTATTGCCTACAATACGCGTCAAGTCCCTACACATTTGGCGGG | | |
| | GGAACAAAGCTGGAAATCAAGCGTACGGTGGCTGCACCATCTGTCTTCATCTTCCCGCCA | | |
| | TCTGATGAGCAGTTGAAATCTGGAACTGCCTCTGTTGTGTGCCTGCTGAATAACTTCTAT | | |
| | CCCAGAGAGGCCAAAGTACAGTGGAAGGTGGATAACGCCCTCCAATCGGGTAACTCCCAG | | |
| | GAGAGTGTCACAGAGCAGGACAGCAAGGACAGCACCTACAGCCTCAGCAGCACCCTGACG | | |
| | CTGAGCAAAGCAGACTACGAGAAACACAAAGTCTACGCCTGCGAAGTCACCCATCAGGGC | | |
| | CTGAGCTCGCCCGTCACAAAGAGCTTCAACAGGGGAGAGTGT | | |
| Nucleotide | ATGGGATGGTCATGTATCATCCTTTTTCTGGTAGCAACTGCAACTGGAGTACATAGCGAG | | 29 |
| sequence of | GTCCAGTTAGTGCAATCGGGAGCCGAAGTCAAGAAGCCAGGTGCCTCCGTTAAAGTTTCA | | |
| IGT-427 HC | TGTAAAGCTTCAGGATACAGTTTCACATCTTATTGGATGAATTGGGTTAGACAGGCTCCA | | |
| with IgG₁ | GGTCAAGGCCTGGAGTGGATGGGCATGATCCATCCTTCTGATAGCGAAACCCGCCTGAAC | | |
| linker | CAGAAGTTCATGGACAGAGTTACCATGACCCGGGACACCAGCACTAGCACCGTATACATG | | |
| | GAGCTGAGTAGCCTGAGGTCAGAGGATACAGCCGTGTATTACTGTGCACGTGGCCTTTAT | | |
| | GGCAATTCTTGGGGCCAGGGCACTTTGGTAACAGTCTCCTCAGCTAGCACCAAGGGCCCA | | |
| | TCGGTCTTCCCCCTGGCACCCTCCTCCAAGAGCACCTCTGGGGGCACAGCGGCCCTGGGC | | |
| | TGCCTGGTCAAGGACTACTTCCCCGAACCGGTGACGGTGTCGTGGAACTCAGGCGCCCTG | | |
| | ACCAGCGGCGTGCACACCTTCCCGGCCGTCCTACAGTCCTCAGGACTCTACTCCCTCAGC | | |
| | AGCGTGGTGACCGTGCCCTCCAGCAGCTTGGGCACCCAGACCTACATCTGCAACGTGAAT | | |
| | CACAAGCCCAGCAACACCAAGGTGGACAAGAAGGTTGAGCCCAAATCTTGTGACAAAACT | | |
| | CACACATGCCCACCGTGCCCAGCACCTGAAGCCGCTGGGGGACCGTCAGTCTTCCTCTTC | | |
| | CCCCCAAAACCCAAGGACACCCTCATGATCTCCCGGACCCCTGAGGTCACATGCGTGGTG | | |
| | GTGGACGTGAGCCACGAAGACCCTGAGGTCAAGTTCAACTGGTACGTGGACGGCGTGGAG | | |
| | GTGCATAATGCCAAGACAAAGCCGCGGGAGGAGCAGTACAACAGCACGTACCGTGTGGTC | | |
| | AGCGTCCTCACCGTCCTGGCCCAGGACTGGCTGAATGGCAAGGAGTACAAGTGCAAGGTC | | |
| | TCCAACAAAGCCCTCCCAGCCCCCATCGAGAAAACCATCTCCAAAGCCAAAGGGCAGCCC | | |
| | CGAGAACCACAGGTGTACACCCTGCCCCCATCCCGGGACGAGCTGACCAAGAACCAGGTC | | |
| | AGCCTGACCTGCCTGGTCAAAGGCTTCTATCCCAGCGACATCGCCGTGGAGTGGGAGAGC | | |
| | AATGGGCAGCCGGAGAACAACTACAAGACCACGCCTCCCGTGCTGGACTCCGACGGCTCC | | |
| | TTCTTCCTCTACAGCAAGCTCACCGTGGACAAGAGCAGGTGGCAGCAGGGGAACGTCTTC | | |
| | TCATGCTCCGTGATGCATGAGGCTCTGCACAACCACTACACGCAGAAGAGCCTCTCCCTG | | |
| | TCTCCGGGCAAAGGAGGAGGCGGCTCTGGTGGTGGAGGCAGTGAACCCAAGAGCAGCGAC | | |
| | AAGACACATACAAGTGACACAGGCCGGCCATTCGTGGAAATGTATTCCGAAATACCTGAG | | |
| | ATCATTCACATGACAGAGGGCAGAGAGCTGGTCATTCCGTGTCGGGTCACATCCCCTAAT | | |
| | ATCACTGTGACCTTGAAAAAGTTCCCCCTGGATACCCTGATTCCTGACGGAAAGCGCATC | | |
| | ATCTGGGACAGCAGGAAAGGGTTCATCATCTCTAACGCCACCTACAAGGAAATTGGCTTG | | |
| | CTGACATGTGAAGCAACCGTCAACGGGCACCTGTACAAGACCAACTATCTGACTCATCGG | | |
| | CAAACTAATACCATCATCGATGTGGTGCTCTCTCCAAGTCATGGAATTGAGCTCTCCGTT | | |
| | GGCGAGAAACTGGTGCTCAATTGCACCGCACGGACGGAGTTGAACGTCGGAATCGATTTT | | |
| | AATTGGGAGTACCCCTCCTCGAAGCATCAGCACAAGAAGCTCGTAAACCGAGATCTCAAA | | |
| | ACCCAGTCCGGCTCTGAGATGAAGAAATTCCTGAGCACACTGACAATTGATGGAGTGACC | | |
| | CGAAGCGACCAGGGCCTCTACACCTGCGCTGCTAGCTCCGGATTGATGACCAAGAAGAAC | | |
| | TCCACTTTCGTCCGAGTGCACGAGAAG | | |
| IGT-427 CL1 | KRTVAAPSVFIFPPSDEQLKSGTASVVCLLNNFYPREAKVQWKVDNALQSGNSQESVTEQ | | 30 |
| Domain | DSKDSTYSLSSTLTLSKADYEKHKVYACEVTHQGLSSPVTKSFNRGEC | | |

### DESCRIPTION OF THE EMBODIMENTS

### Definitions

Unless stated otherwise, the following terms and phrases as used herein are intended to have the following meanings:

As used herein, the term “anti-Tie2 antibody” means an antibody specifically binding to Tie2 and includes, in addition to a complete antibody specifically binding to Tie2, antigen-binding fragments of the antibody molecule. In some embodiments, a complete antibody has the structure of two full-length light chains and two full-length heavy chains, and each light chain is connected to a heavy chain by a disulfide bond. The constant region of the heavy chain has gamma (γ), mu (μ), alpha (α), delta (δ) and epsilon (ε) types, with the subclasses of Gamma 1 (γ1), Gamma 2 (γ2), Gamma 3 (γ3), Gamma 4 (γ4), Alpha 1 (α1) and alpha 2 (α2). The constant region of the light chain has kappa (κ) and lambda (λ) types.

“Antigen-binding fragments” or “antibody fragments of an antibody” means a fragment which can bind to an antigen, and includes Fab, F(ab′), F(ab′)2 and Fv. Among antibody fragments, Fab has one antigen-binding site with a structure of the variable regions of the light and heavy chain, the constant region of the light chain, and the first CH1 of the heavy chain.

Fab′ differs from Fab in that it has a hinge region comprising one or more cysteine residues at the C-terminus of the CH1 domain. F(ab′)2 antibody is produced by disulfide bonds formation between Cysteine residues in the region of the hinge of Fab′. Fv is the smallest antibody fragment having only the variable region of the heavy chain and the variable region of the light chain Double chain Fv (two-chain Fv) is formed by a non-covalent bond between the heavy chain variable region and the light chain variable region, and single-chain Fv (scFv) is generally formed through a peptide linker covalently between the variable region of the heavy chain and the variable region of the light chain, or is connected directly at the C-terminus by forming a dimer-like structure like a double-chain Fv. This fragment can be obtained by protein hydrolysis enzyme (e.g., one can get Fab by restriction digestion of whole antibody using papain, one can get F(ab′)2 fragment by cutting with pepsin), also made by genetic manipulation technology.

An antibody may be, for example, in the Fv form (e.g., scFv), or a complete antibody form. In addition, constant region of the heavy chain may be selected from any isotypes of gamma (γ), mu (μ), alpha (α), delta (δ), or epsilon (ε). For example, the constant region is gamma 1 (IgG1), gamma 3 (IgG3), or gamma 4 (IgG4). The light chain constant region can be of kappa or lambda type.

Antibodies include monoclonal antibodies, multispecific antibodies, human antibodies, humanized antibodies, chimeric antibodies, single chain Fvs (scFV), single chain antibodies, Fab fragments, F(ab′) fragments, Disulfide-binding Fvs (sdFV) and anti-idiotype (anti-Id) antibodies, or of the above antibodies epitope-binding fragments and the like, but are not limited thereto.

The term, “heavy chain” of “HC”, means the full-length heavy chain or fragments thereof comprising a variable region domain VH and three constant region domains CH1, CH2 and CH3, having an amino acid sequence with a sufficient variable region in order to provide antigen specificity. In addition, as used herein, “light chain” or “LC”, means the full-length light chain or fragments thereof comprising a variable region domain VL and a constant region domain CL, having an amino acid sequence with a sufficient variable region in order to provide antigen specificity. The heavy chain constant region domains (e.g., of a human IgG1) can contain one or more modifications to reduce or eliminate efficient interaction with Fc Receptors on immune cells in the body. Such mutation(s) can abrogate its binding to Fc receptors and reduce or abolish antibody-directed cytotoxicity. Such modifications can include altering the L₂₃₄L₂₃₅G₂₃₆G₂₃₇ residues (EU numbering) to a LAGA mutation, a FEGG mutation, an AAGG mutation, an AAGA mutation, a LALA mutation or a combination thereof. In some embodiments, an IgG constant region comprises mutations at K322 and P331 (EU numbering) to reduce or abolish the immune cell-mediated cytotoxicity (e.g., in combination with any of the foregoing mutations). In some embodiments, an IgG constant region comprises a LALA mutation as well as mutations at K322 and P331 (e.g., K322A and P331S) (EU numbering) to reduce or abolish the immune cell-mediated cytotoxicity.

A “monoclonal antibody” is an antibody produced by a single clone of cells or cell line and consisting of identical antibody molecules. Monoclonal antibodies can have monovalent affinity, binding only to the same epitope. In contrast, polyclonal antibodies bind to multiple epitopes and are usually made by several different antibody secreting plasma cell lineages. Bispecific monoclonal antibodies can also be engineered, by increasing the targets of one monoclonal antibody to two epitopes.

“Epitope” means a protein determinant to which an antibody can specifically bind to (e.g., the part of an antigen that is recognized by the antibody). An epitope can be a group of chemically active surface molecules, e.g., amino acids or sugar side chains, and generally has a specific charge characteristic as well as a specific three-dimensional structural characteristic.

The “humanized” form of non-human (e.g., murine) antibody is a chimeric antibody comprising one or more amino acid sequence (e.g., one or more CDR sequences, such as 6 CDR sequences) from a non-human antibody (donor or source antibody) and otherwise minimal sequence derived from non-human immunoglobulins. In some embodiments, the humanized antibody is a human immunoglobulin (receptor antibody) whose hypervariable regions are replaced by residues from hypervariable regions of non-human primates, mouse, rat, rabbit or non-human primate (receptor antibody), possessing the desired specificity, affinity and ability of residues from the hypervariable region of the recipient. For humanization one or more residues in the framework domain (FR) can be replaced by the corresponding residue of the non-human donor antibody. This can help to maintain a proper three-dimensional configuration of the grafted CDR(s), thereby improving affinity and antibody stability. Humanized antibodies, e.g., can alternatively or additionally include a new residue that does not appear in the original recipient antibody or donor antibody to further refine additional performance of antibody.

Any “chimeric” antibodies (immunoglobulins) as well as the fragment of the above-mentioned antibody, which exhibit the desired biological activity, are included where part of the heavy and/or light chain derived from a particular species, or identical or homologous to the corresponding sequence in the antibody belonging to the subclass, while the remaining chain(s) are derived from another species, or belonging to other antibody classes or identical to the corresponding sequence in the antibody belonging to the subclass.

“Antibody variable domain” as used herein refers to a domain comprising complementarity determining regions (CDRs; e.g., CDR1, CDR2, and CDR3) and the framework regions (FRs). VH refers to the variable domain of the heavy chain VL refers to the variable domain of the light chain. “Framework region” (FR) is a variable domain segment outside of the CDRs. Each variable domain typically has 4 FRs identified as FR1, FR2, FR3 and FR4.

“Complementarity determining regions” (CDRs; i.e., CDR1, CDR2, and CDR3) refers to the amino acid residues of the variable domain of the antibody, which are necessary for antigen binding. Each variable domain typically, comprises three CDR regions identified as CDR1, CDR2 and CDR3.

A “half-life extension modulator,” as used herein, refers to chemicals, biopolymers, peptides, polypeptides, or protein fragments that can be added to the antibody or antibody fragments to increase its half-life. The half-life extension modulators may include: biopolymers which contain PEG (polyethylene-glycol), hyaluronic acid (HA), or phosphorylcholine; albumin; albumin-binding peptide; or HA-binding protein fragments.

A half-life may also be decreased, for example, by abolishing the interaction of the antibody or antibody fragment with neonatal Fc receptor (FcRn)-mediated recycling. For example, combinatorial mutations at multiple sites are known to affect an antibody's half-life. In some embodiments, the mutations include M252 (e.g., M252Y), I253 (e.g., I253A, I253M, or I253V), S254 (e.g., S254T), T256 (e.g., T256D), H310 (e.g., H310A), H433 (e.g., H433K), N434 (e.g., N434F), and/or H435 (e.g., H435A, H435Q or H435R) (EU numbering). In some embodiments, the mutations include L235A, L236A, and H310A (EU numbering). A decrease in systemic half-life may be advantageous with non-systemic disease treatment, such as ocular disease treatment, where minimal systemic exposure and decreased systemic half-life are desired due to established on-target toxicity issues of VEGF inhibition (bleeding, hypertension, etc.).

As used herein, “or” has the inclusive sense (i.e., equivalent to and/or) unless the context clearly indicates otherwise.

As used herein, “fusion protein” encompasses any polypeptide comprising sequences from multiple sources. Fusion proteins may be produced, e.g., from a genetic fusion (e.g., a polynucleotide encoding the sequence of the fusion protein) or by chemically joining polypeptides that were produced or synthesized separately.

“Angiogenic disease,” also referred to as “angiogenesis-related disease,” means the occurrence of angiogenesis or a disease associated with progression of angiogenesis.

As used herein, the “subject” may be a human or an animal. For example, the subject may be a human. In some embodiments, the subject may be a mammal. In some embodiments, the subject may include a companion animal (also referred to as a “pet”). The term “companion animal” refers to a domesticated animal that can be kept as a pet or otherwise for companionship purposes and includes, but is not limited, to dogs, cats, rabbits, ferrets, horses, donkeys, mules, and hamsters. In some embodiments, a companion animal is a mammalian companion animal.

The term “prevention” as used herein denotes any action to inhibit, delay the onset of, or reduce the likelihood of occurrence of the disease of interest by administering the fusion protein or composition. The terms “treatment” or “therapy” indicate any action that reduces, delays, or mitigates the symptoms of the disease of interest, or cures, reduces the severity of, or delays the progression of the disease of interest.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to said given sequence.

As used herein, the term “about” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### Overview

Provided herein are fusion proteins that specifically bind to both Tie2 and VEGF. The fusion proteins were designed to address both limitations of the current therapies of Tie2 and VEGF, as well as the limitations of other currently available therapies for angiogenic or vascular disease. As a result, the fusion proteins are provided comprising an anti-Tie2 antibody or antigen-binding fragment thereof and a vascular endothelial growth factor (VEGF)-binding domain. These fusion proteins can play a beneficial role as therapeutic agents for angiogenic or vascular diseases due to their dual function of VEGF inhibition and Tie2 activation.

### Fusion Proteins

The present disclosure relates to a fusion protein that comprises an Tie2 antibody or antigen-binding fragment thereof and a VEGF-binding domain. In some embodiments, the Tie-2 antibody or antigen-binding fragment thereof binds to the Tie2 Ig3-FNIII (1-3) domain comprising the sequence of SEQ ID NO: 2.

In some embodiments, the Tie2 antibody or antigen-binding fragment thereof includes a heavy chain variable region comprising heavy chain CDR1, CDR2, and CDR3 having the amino acid sequences of SEQ ID NOs: 5-7, respectively, and a light chain variable region comprising light chain CDR1, CDR2, and CDR3 having the amino acid sequences of SEQ ID NOs: 8-10.

In some embodiments, the fusion protein binds to a Tie2 Ig3-FNIII (1-3) domain comprising SEQ ID NO: 2, 3, or 4 and to VEGF.

In some embodiments, the fusion protein comprises a vascular endothelial growth factor (VEGF)-binding domain, the VEGF-binding domain comprising a vascular endothelial growth factor (VEGF)-A binding region of VEGF receptor 1 (VEGFR1) of SEQ ID NO: 13 and a vascular endothelial growth factor (VEGF)-A binding region of VEGF receptor 2 (VEGFR2) of SEQ ID NO: 14, and an anti-Tie2 antibody or antibody binding fragment thereof, wherein the fusion protein binds to Tie2 Ig3-FNIII (1-3) domain comprising SEQ ID NO: 2, 3, or 4, and VEGF.

In some embodiments, the VEGF-binding domain comprises an anti-VEGF antibody or antibody binding fragment thereof. In a further embodiment, the anti-VEGF antibody or fragment thereof comprises the variable binding domains of bevacizumab, ranibizumab or HuMab G6-31 (US Patent Publication No. 2007/0141065).

In one embodiment, the VEGF-binding domain is linked to the C-terminus of the heavy chain (HC) of the anti-Tie2 antibody or antigen-binding fragment thereof.

In one embodiment, the fusion protein binds amino acids 633-644 (SEQ ID NO: 20) and/or amino acids 713-726 (SEQ ID ON: 21) of Tie2 having the amino acid sequence of SEQ ID NO:1. In one embodiment, the anti-Tie2 antibody or antibody binding fragment thereof binds amino acids 633-644 (SEQ ID NO: 19) and/or amino acids 713-726 (SEQ ID NO: 20) of Tie2 of the amino acid sequence of SEQ ID NO:1.

In one embodiment, the anti-Tie2 antibody has an IgG1 isotype.

In one embodiment, the VEGF-binding domain comprises a sequence having at least 80%, 85%, 90%, 95%, 96%, 97%, 98%, or 99% identity to the amino acid sequence of SEQ ID NO: 15. In one embodiment, the VEGF-binding domain comprises the amino acid sequence of SEQ ID NO: 15.

In one embodiment, the fusion protein comprises four polypeptide chains, wherein two chains are a pair of heavy chains, and two chains are a pair of light chains. Suitably, the heavy chains comprise at the N-terminal end a Tie2-binding domain which binds to Tie2 Ig3-FNIII (1-3) domain comprising SEQ ID NO: 2, 3, or 4, and at the C-terminal end a VEGF-binding domain. Suitably the Tie2-binding domain is an anti-Tie2 antibody, wherein the pair of heavy chains of the fusion protein comprise the heavy chain of the antibody and the pair of light chains of the fusion protein comprise the light chains of the antibody.

In one embodiment, the fusion protein comprises
a. a dimer of a first and second heavy chain monomer, wherein each heavy chain monomer comprises a single-chain polypeptide comprising, from N-terminus to C-terminus:
   i. a heavy chain variable domain (VH) domain that together with a light chain variable domain binds Tie2, linked to
   ii. a constant heavy chain domain (CH) comprising an CH1 domain and an Fc region or fragment thereof, linked to
   iii. a VEGF-binding domain comprising a VEGF receptor extracellular domain, (such as VEGF-A binding regions of a VEGF Receptor 1 (VEGFR1) and a VEGF Receptor 2 (VEGFR2)); and
b. a first and second light chain monomer, each light chain monomer comprising from N-terminus to C-terminus a light chain variable domain (VL) that together with a VH binds Tie2, linked to a constant light chain domain (CL1);
wherein the first and second monomers dimerize via their Fc regions, or fragments thereof;
and wherein the Tie2-binding domains are formed by the pairing of each heavy chain monomer with one of said light chain monomer such that the VH and CH1 of each heavy chain monomer pairs with the VL and CL1 domain of the light chain monomers.

In one embodiment, the fusion protein comprises a linker between the VEGF receptor and the anti-Tie2 antibody or antibody fragment thereof. In a further embodiment, the linker comprises between 5 and 50 residues, such as between 10 and 40 residues, such as between 15 and 30 residues, such as 20 residues.

In some embodiments, the fusion protein comprises a linker between the VEGF-binding domain and the anti-Tie2 antibody or antibody fragment thereof, and wherein the linker comprises a sequence having at least 80%, 85%, 90%, 95%, 96%, 97%, 98%, or 99% identity to the amino acid sequence of SEQ ID NO: 16. In some embodiments, the linker comprises the sequence of SEQ ID NO: 16.

In some embodiments, the fusion protein comprises a linker between the VEGF receptor and the anti-Tie2 antibody or antibody fragment thereof, and wherein the linker comprises a sequence having at least 80%, 85%, 90%, 95%, 96%, 97%, 98%, or 99% identity to the amino acid sequence of SEQ ID NO: 25. In some embodiments, the linker comprises the sequence of SEQ ID NO: 25.

In one embodiment, the fusion protein comprises, C-terminal to the heavy chain constant domain or domains of the anti-Tie2 antibody or antigen-binding fragment thereof and in N- to C-terminal order, a linker between the VEGF-binding domain and the anti-Tie2 antibody or antibody fragment thereof, such as wherein the linker comprises a sequence having between 5 and 50 amino acid residues, such as between 10 and 40 residues, such as between 15 and 30 residues, such as 20 residues.

In one embodiment, the fusion protein comprises, C-terminal to the heavy chain constant domain or domains of the anti-Tie2 antibody or antigen-binding fragment thereof and in N- to C-terminal order, a linker between the VEGF-binding domain and the anti-Tie2 antibody or antibody fragment thereof, wherein the linker comprises amino acid sequence of SEQ ID NO: 16 or 25, and the VEGF-binding domain comprises the amino acid sequence of SEQ ID NO: 15.

In one embodiment, the fusion protein comprises a CH domain comprising the amino acid sequence of SEQ ID NO: 17, a linker comprising amino acid sequence of SEQ ID NO: 16 or 25 between the VEGF-binding domain and the anti-Tie2 antibody or antibody fragment thereof, and the VEGF-binding domain comprises the amino acid sequence of SEQ ID NO: 15.

In one embodiment, the fusion protein comprises a heavy chain variable region comprising the amino acid sequence of SEQ ID NO:18 and a light chain variable region comprising the amino acid sequence of SEQ ID NO:19.

In one embodiment, the fusion protein comprises a heavy chain comprising at least 80%, 85%, 90%, 95%, 96%, 97%, 98%, or 99% identity to the amino acid sequence of SEQ ID NO: 11. In one embodiment, the fusion protein comprises a heavy chain comprising the amino acid sequence of SEQ ID NO:11.

In one embodiment, the fusion protein comprises a heavy chain comprising at least 80%, 85%, 90%, 95%, 96%, 97%, 98%, or 99% identity to the amino acid sequence of SEQ ID NO: 26. In one embodiment, the fusion protein comprises a heavy chain comprising the amino acid sequence of SEQ ID NO: 26.

In one embodiment, the fusion protein comprises a light chain comprising at least 80%, 85%, 90%, 95%, 96%, 97%, 98%, or 99% identity to the amino acid sequence of SEQ ID NO: 12. In one embodiment, the fusion protein comprises a light chain comprising amino acid sequence of SEQ ID NO:12. In one embodiment, the fusion protein comprises a heavy chain comprising amino acid sequence of SEQ ID NO:11, and a light chain comprising amino acid sequence of SEQ ID NO:12.

In one embodiment, the fusion protein comprises a CH domain comprising a sequence having at least 80%, 85%, 90%, 95%, 96%, 97%, 98%, or 99% identity to the amino acid sequence of SEQ ID NO: 17. In one embodiment, the fusion protein comprises a CH domain comprising the amino acid sequence of SEQ ID NO: 17. In some embodiments, the fusion proteins comprises one or more modifications or mutations in the constant region domains in the heavy chain to reduce or eliminate efficient interaction with Fc Receptors on other immune cells in the body constant region domains. In some embodiments, the one or more modifications or mutations comprises altering L₂₃₄L₂₃₅G₂₃₆G₂₃₇ (EU numbering) to a LAGA mutation, a FEGG mutation, an AAGG mutation, an AAGA mutation, a LALA mutation, or a combination thereof. In some embodiments, an IgG constant region comprises any of the foregoing mutations (e.g., a LALA mutation) as well as mutations at K322 and P331 (EU numbering) to reduce and abolish the immune cell-mediated cytotoxicity.

Tie2 antibodies are monovalent or bivalent, and contain single or double chains. Functionally, the binding affinity, or dissociation constant (K_{D}), is the molar (M) concentration of ligand at which half the ligand binding sites on the protein are occupied in the system equilibrium. It is calculated by dividing the dissociation rate (K_{off}) value by the association rate (Kₒₙ) value. In the context of dissociation constant, a lower value is consistent with stronger binding. The K_{D} of the Tie2 antibody is in the range of 10⁻⁵M to 10⁻¹²M. For example, the binding affinity (K_{D}) of the Tie2 antibody is 10⁻⁶ M to 10⁻¹²M, 10⁻⁷ M to 10⁻¹² M, 10⁻⁸ M to 10⁻¹² M, 10⁻⁹ M to 10⁻¹² M, 10⁻⁵ M to 10⁻¹¹ M, 10⁻⁶ M to 10⁻¹¹ M, 10⁻⁷ M to 10⁻¹¹ M, 10⁻⁸ M to 10⁻¹¹ M, 10⁻⁹ M to 10⁻¹¹ M, 10⁻¹⁰ M to 10⁻¹¹ M, 10⁻⁵ M to 10⁻¹⁰ M, 10⁻⁶ M to 10⁻¹⁰ M, 10⁻⁷ M to 10⁻¹⁰ M, 10⁻⁸ M to 10⁻¹⁰ M, 10⁻⁹ M to 10⁻¹⁰ M, 10⁻⁵ M to 10⁻⁹ M, 10⁻⁶ M to 10⁻⁹ M, 10⁻⁷ M to 10⁻⁹ M, 10⁻⁸ M to 10⁻⁹ M, 10⁻⁵ M to 10⁻⁸ M, 10⁻⁶ M to 10⁻⁸ M, 10⁻⁷ M to 10⁻⁸M, 10⁻⁵ M to 10⁻⁷ M, 10⁻⁶ M to 10⁻⁷M or 10⁻⁵M to 10⁻⁶ M.

IGT-427, an exemplary fusion protein described herein, simultaneously suppresses VEGF signaling and activates Tie2 signaling pathways. IGT-427 binds human Tie-2 with K_{D}<1 nM in a bivalent interaction, analogous to the cellular surface interaction, and binds human VEGF with a K_{D}<10 pM. With VEGF on the surface, IGT-427 binds with an apparent affinity of <500 pM. With IGT-427 on the surface, the K_{D} is <10 pM. IGT-427 displays comparable high affinities toward rabbit orthologs.

In one embodiment, the fusion protein binds to Tie2 Ig3-FNIII (1-3) domain comprising SEQ ID NO: 2, 3, or 4 with an affinity K_{D} (M) of less than 3E⁻⁹ M (where E^{-X} denotes negative exponent and the number of zeros, e.g., 3E⁻⁹ is the same as 0.0000000003), less than 3.5E⁻⁹ M, less than 4E⁻¹⁰ M, or less. A K_{D} value less than a given amount means that the K_{D} is numerically smaller than the given amount, which indicates stronger binding.

IGT-427 has the potential to provide superior efficacy compared to agents that only inhibit VEGF, and/or to have more potent Tie-2 agonist activity compared to Ang2 inhibiting agents.

The Tie2 antibody or antibody fragment in the fusion protein can include its biological equivalent within a range that can specifically recognize Tie2. For example, it can include a change to the amino acid sequence to improve further the binding affinity and/or its other biological properties of the antibody. Such modifications can include, for example, deletion, insertions and/or substitutions of amino acid sequence residues of the antibody. These amino acid variations can be made based on the relative similarity of the amino acid substituents, such as hydrophobicity, hydrophilicity, charge, size, etc. of the amino acid side chains, so as to provide conservative substitutions. By the analysis of the size, shape and type of amino acid side chain substituents, it is known that arginine, lysine and histidine are all positively charged residues; alanine, glycine and serine have similar sizes; Phenylalanine, tryptophan and tyrosine have a similar shape. Therefore, based on these considerations, we can say that arginine, lysine and histidine; alanine, glycine and serine; and phenylalanine, tryptophan and tyrosine are conservative substitutions.

Considering the above-described variation having biologically equivalent activity, in some embodiments, the amino acid sequence of the fusion protein includes the sequences of the six CDRs in SEQ ID NOs: 5-10, and/or the heavy and light chains of SEQ ID NOs: 11 and 12, or any sequence exhibiting substantial identity. Substantial identity means at least 80% identity, such as at least 85% identity, 90% identity, 95% identity, 96% or more, 97% or more, 98% or more, or 99% or more sequence identity.

Based on this, the fusion protein, or components thereof, may have 80%, 85%, 90%, 91%, 92%, 93%, 94%, 95%, 96%, 97%, 98%, 99%, or more identity, when compared to the specified or all sequence described in the specification.

In one embodiment, the fusion protein is pegylated. In one embodiment, the fusion protein is modified with a half-life extension modulator, such as PEG, hyaluronic acid, or phosphorylcholine. In one embodiment, the fusion protein is site-specifically pegylated. In one embodiment, the fusion protein is site-specifically pegylated on a cysteine residue. In one embodiment, the fusion protein is site-specifically modified (e.g., on a cysteine residue) with a half-life extension modulator, which may comprise PEG, hyaluronic acid, or phosphorylcholine. Alternatively, the half-life extension modulator may comprise an albumin; an albumin-binding peptide, and/or an HA-binding protein fragment.

In one embodiment, the fusion protein further comprises the sequence of SEQ ID NO: 22 and is site-specifically modified with any of the half-life extension modulators described herein (e.g., pegylated) on the cysteine residue of the sequence of SEQ ID NO: 22. In one embodiment, the sequence of SEQ ID NO: 22 is present at the C-terminus of the heavy chain. In one embodiment, the heavy chain comprises the sequence of SEQ ID NO: 23. In one embodiment, the heavy chain comprises the sequence of SEQ ID NO: 24.

In one embodiment, the PEG has a molecular weight of about 40 kDa.

In one embodiment, there is provided a polypeptide comprising a chain monomer of the fusion protein of the invention.

In one embodiment, the polypeptide comprises the heavy chain monomer of the fusion protein of the invention. In a further embodiment, the polypeptide comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to the sequence of SEQ ID NO: 11 or 26. In one embodiment, the polypeptide consists of the sequence of SEQ ID NO: 11. In one embodiment, the polypeptide consists of the sequence of SEQ ID NO: 26.

In one embodiment, the polypeptide comprises the light chain monomer of the fusion protein of the invention. In a further embodiment, the polypeptide comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to SEQ ID NO: 12. In one embodiment, the polypeptide consists of the sequence of SEQ ID NO: 12.

### Polynucleotides; Methods of Production; Vectors and Host Cells

In another aspect, the present disclosure relates to nucleic acids encoding the fusion protein.

In another embodiment, the disclosure provides a nucleic acid encoding a polypeptide comprising a chain monomer of the fusion protein of the invention. In one embodiment, the disclosure provides a nucleic acid encoding the heavy chain of the fusion protein. In another embodiment, the disclosure provides a nucleic acid encoding the light chain of the fusion protein.

In one embodiment, the nucleic acid molecule comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to SEQ ID NO: 27 or 29. In one embodiment, the nucleic acid molecule consists of SEQ ID NO: 27 or 29.

In one embodiment. the nucleic acid molecule comprises a sequence which has at least 70% identity, such as at least 75% identity, such as at least 80% identity, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity, such as 100% identity to SEQ ID NO: 28. In one embodiment, the nucleic acid molecule consists of SEQ ID NO: 28.

In one embodiment, the disclosure provides a set of one or more polynucleotides wherein each polynucleotide encodes at least one of the monomer chains of the fusion protein of the invention, such both chains (i.e., light and heavy chains) of said fusion protein are encoded.

The fusion protein can be produced recombinantly by constructing or isolating the nucleic acid encoding the fusion protein. Further cloning (DNA amplification) by isolating nucleic acids, and inserting it into a replicable vector may be done or further expression may be made. Based on this, the present disclosure relates to the vector containing the nucleic acid in another aspect.

In some embodiments, the disclosure relates to a nucleic acid comprising the fusion protein. “Nucleic acid” is meant to encompass DNA (gDNA and cDNA) and RNA molecules inclusively, and nucleotide, the basic structural unit of nucleic acid, includes the nucleotide in nature, as well as the analogue with modified sugar or base moieties. The sequence of the nucleic acid encoding the heavy and light chain variable regions can be modified. The modifications include addition, deletion, or non-conservative or conservative substitution of nucleotides.

The DNA encoding the fusion protein is easily separated or synthesized using a conventional process (for example, by using oligonucleotide probes capable of specifically binding to the DNA encoding the heavy and light chains) Many vectors are available. Vector components generally include one or more of the following, but is not limited to: signal sequence, origin of replication, one or more marker genes, enhancer element, promoter, and transcription termination sequence.

In some embodiments, the disclosure provides an expression vector comprising the nucleic acid encoding the fusion protein. In another embodiment, there is provided a vector which comprises nucleic acids encoding one heavy chain sequence and one light chain sequence of the fusion protein.

In one embodiment, there is provided a set of one or more vectors which collectively comprise the set of one or more polynucleotides of the invention, such that both chains (i.e., light and heavy chains) of said fusion protein are encoded in the set of vectors.

In one embodiment, the vector is an animal virus, such as a virus selected from reverse transcriptase virus (including lentivirus), adenovirus, adeno-associated virus, herpes virus, chicken pox virus, baculovirus, papilloma virus, and papova virus.

In some embodiments, the expression vector further comprises a promoter/enhancer, such as a human cytomegalovirus IE1 (CMV-IE1) promoter/enhancer.

As used herein, the term “vector”, as means for expression a gene of interest in a host cell, includes plasmid vectors, cosmid vector, bacteriophage vector, adenovirus vectors, retroviral vectors and viral vectors such as adeno-associated virus vectors. The nucleic acid encoding the antibody in the vector is operatively linked to a promoter.

As used herein, the terms “operatively linked” or “linked” mean the functional linkage between a nucleic acid expression control sequence (for example, promoter, signal sequence, or array of transcriptional regulator binding sites) and different nucleic acid sequences, Thereby, the control sequence controls transcription and/or translation of the other nucleic acid sequence.

In some embodiments, provided herein is a host cell comprising a polynucleotide encoding a fusion protein described herein. In some embodiments, provided herein is a host cell comprising a vector comprising a polynucleotide encoding a fusion protein described herein. The host cell may be prokaryotic or eukaryotic. The host cell may be isolated, e.g., cultured or not part of a multicellular organism. In some embodiments, the host cell is a member of a cell line. In some embodiments, the host cell is a mammalian cell. In some embodiments, the host cell is an immortalized mammalian cell.

In the case of prokaryotic cell as a host, a strong promotor which can process the transcription (for example, tac promoter, lac promoter, lacUV5 promoter, 1 pp promoter, pLλ promoter, pRλ promoter, rac5 promoter, amp promoter, recA promoter, SP6 Promoter, trp promoter and T7 promoter, etc.), ribosome binding site for initiation of translation and transcription/translation termination sequences are generally included. In addition, for example, in the case of eukaryotic cell as a host, a promoter derived from the genome of mammalian cells (example: metallothionine promoter, β-actin promoter, human hemoglobin promoter and human muscle creatine promoter) or a promotor derived from mammalian viruses (example: adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, Moloney Virus promotor, Epstein Barr Virus (EBV) promoter, and Rous sarcoma virus (RSV) promoter) can be used, and a polyadenylation sequence can be included generally as a transcription termination sequence.

In some cases, the vector may be fused with other sequences to facilitate the purification of the antibody expressed. The sequence to be fused is, for example, glutathione S-transferase (Pharmacia, USA), maltose binding protein (NEB, USA), FLAG (IBI, USA) and 6×His (hexahistidine; Qiagen, USA).

The vector contains an antibiotic resistance gene commonly used in the art as a selection marker, for example a resistant gene to ampicillin, gentamicin, carbenicillin, chloramphenicol, streptomycin, kanamycin, geneticin, neomycin and tetracycline.

In another aspect, the present disclosure relates to a cell transformed with the above-mentioned expression vector. Cells used to produce the antibodies may be prokaryote, yeast and a higher eukaryotic cell, but not limited thereto.

Prokaryotic host cells such as Escherichia coli, Bacillus strains such as Bacillus subtilis and Bacillus thuringiensis, Streptomyces, Pseudomonas (e.g., Pseudomonas putida), Proteus mirabilis and Staphylococcus (for example, Staphylococcus carnosus), can be used.

Exemplary useful animal host cell lines are COS-7, BHK, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TM, MRC 5, FS4, 3T3, MN, A549, PC12, K562, PER.C6, SP2/0, NS-0, U205, or HT1080, but not limited thereto.

In one embodiment, the disclosure provides a method of manufacturing a fusion protein which binds Tie2 and VEGF, comprising the steps of: culturing a cell comprising a nucleic acid, such as an expression vector, encoding the fusion protein (e.g., transformed with an expression vector comprising a nucleic acid encoding the fusion protein); and recovering a fusion protein from the cultured cell.

The cells can be cultured in various media. Any commercial culture media can be used without limitation. All other essential supplements known to those skilled in the art may be included in an appropriate concentration. Culture conditions, such as temperature, pH, etc., and selected host cells are known to those skilled in the art.

Recovery of the antibody or antigen-binding fragment thereof can be made through removing impurities for example using centrifugation or ultrafiltration, and for example, using affinity chromatography, etc. Additional extra purification technology such as anion or cation exchange chromatography, hydrophobic interaction chromatography, hydroxyapatite chromatography, and the like can be used.

### Use of Fusion Proteins; Therapy

In another aspect, the present disclosure relates to methods and compositions for preventing or treating angiogenic or vascular diseases by administering an effective amount of the fusion protein. In other aspects, the present embodiments relate to methods and compositions for regulating angiogenesis, endothelial signaling, inflammation, and/or vascular leakage by administering the fusion protein to a subject in need thereof. In some embodiments, a fusion protein described herein is for use in therapy.

Angiogenesis means the formation or growth of new blood vessels from previously existing blood vessels. Examples of angiogenic or angiogenesis-related diseases include, but are not limited to, cancer, metastasis, diabetic retinopathy, diabetic macular edema, retinopathy of prematurity, corneal graft rejection, macular degeneration, glaucoma such as neovascular glaucoma, systemic erythrosis, proliferative retinopathy, psoriasis, hemophilic arthritis, capillary formation in atherosclerotic plaques, keloid, wound granulation, vascular adhesion, rheumatoid arthritis, degenerative osteoarthritis, autoimmune diseases, Crohn's disease, restenosis, atherosclerosis, intestinal adhesions, cat scratch disease, ulcers, liver cirrhosis, nephritis, diabetic nephropathy, diabetes mellitus, inflammatory diseases, and neurodegenerative disease. In addition, exemplary cancers include, but are not limited to esophageal cancer, stomach cancer, large intestine cancer, rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid blood cancer.

The fusion protein may be administered to regulate angiogenesis, endothelial signaling, inflammation, and/or vascular leakage. For example, the fusion protein may be administered to increase vascular endothelial cellular membrane integrity and/or reduce vascular leakage. Vascular leakage is known to contribute to visual impairment in several common ocular disorders including, but not limited to, diabetic macular edema (DME), diabetic retinopathy (DR) and age-related macular degeneration (AMD). In some embodiments, the inflammation is from sepsis, respiratory distress syndromes, and/or virus-infectious diseases.

The fusion protein, and compositions thereof, may be administered into mammals, including rats, mice, livestock, companion animals, humans, etc. In some embodiments, the subject is human. In some embodiments, the subject is a companion animal, such as a mammalian companion animal. In some embodiments, the companion animal is a dog, cat, rabbit, ferret, horse, mule, donkey, or hamster. The fusion protein, or a composition thereof may be administered daily. The fusion protein, or composition thereof may be administered once every 1, 2, 3, 4, 6, or 12 months. In one embodiment, the fusion protein is administered once every six months (i.e., twice a year). Administration is via the typically accepted routes, for example, oral, rectal, intravitreal, intravenous, subcutaneous, intrauterine, or intracerebrovascular administration. In some embodiments, administration is by intravitreal injection. In one embodiment, between 1 mg and 10 mg of fusion protein is administered by intravitreal injection daily, monthly or every six months. In one embodiment, less than 10 mg of fusion protein is administered by intravitreal injection about once every six months (e.g., twice a year).

### Pharmaceutical Compositions; Administration

In some embodiments, the composition comprising the fusion protein is a pharmaceutical composition. In some embodiments, the composition includes a suitable vehicle, excipient or diluent typically used in the field.

Suitable vehicles, excipients, diluents are described in Remington: The Science and Practice of Pharmacy (19th ed.) ed. A. R. Gennaro, Mack Publishing Company, Easton, Pa. 1995. Suitable pharmaceutically acceptable vehicles, excipients or diluents include, for example, one or more of water, saline, phosphate buffered saline, dextrose, histidine, glycerol, sucrose, polysorbate, ethanol and the like, as well as combinations thereof. The pH of the solution is preferably from about 5 to about 8, and more preferably from about 6 to about 7. It will be apparent to those persons skilled in the art that certain carriers may be more preferable depending upon, for instance, the route of administration and concentration.

Pharmaceutical compositions having a pharmaceutically acceptable vehicle can be various oral or parenteral dosage form such as tablets, pills, powders, granules, capsules, suspensions, oral solutions, emulsions, syrups, sterile aqueous solutions, non-aqueous solutions, suspensions, lyophilizates, and suppository. The pharmaceutical composition can include a diluent or excipient, which can be formulated in combination, such as fillers, thickeners, binders, wetting agents, disintegrants, surfactants, etc. Solid preparations for oral administration may be in the form of tablets, pills, powders, granules, capsules, and the like. In connection with the solidarity the compound can be formulated by combining one or more excipients such as starch, calcium carbonate, sucrose, lactose, or gelatin. Simple excipient and lubricating agents such as magnesium stearate, talc, and the like may additionally be used.

The liquid preparation for oral administration may be a suspension, an oral solution, an emulsion, a syrup, or the like. Excipients such as water or simple diluents like wet paraffin, a variety of wetting agents, sweeteners, aromatics, preservatives, and etc. can be included in a liquid formulation. In addition, the pharmaceutical compositions may be in parenteral dosage form such as sterile aqueous solution, non-aqueous solvent, suspension, emulsion, lyophilisate, suppository, etc. Injectable propylene glycol, polyethylene glycol, vegetable oils such as olive oil and esters such as ethyl oleate may be suitable for insoluble solvent and suspension. The basic substance of the suppository includes Witepsol, macrogol, Tween 61, cacao butter, laurin butter and glycerogelatin.

The composition is administered in a pharmaceutically effective amount. Terms used herein, such as a “pharmaceutically effective amount” refers to the enough amount of the pharmaceutical composition for disease treatment, with an appropriate benefit/risk ratio which can be applicable for all medical treatments. The effective amount can be different depending on various factors including parameters like the severity of the disease, the patient's age and sex, type of disease, drug activity, drug sensitivity, administration time, route of administration, secretion rate, duration of treatment, and other factors. Also, the above composition can be administered in single doses or divided into multiple doses. When fully considering these factors, it is important to administer the minimum amount sufficient to obtain maximum effect without side effects. The dosage of the pharmaceutical composition is not particularly limited, but it varies depending on various factors, including patient's health status and weight, disease severity, drug type, administration route and administration time.

The composition may be administered into mammals, including rats, mice, livestock, companion animals, humans, etc., by one time or multiple times a day, via typically accepted routes, for example, orally, rectal, intravenously, subcutaneously, intrauterinely, intravitreally, or intracerebrovascularly. In some embodiments, the composition is administered by intravitreal injection. In some embodiments, the subject is human. In some embodiments, the subject is a companion animal, such as a mammalian companion animal. In some embodiments, the companion animal is a dog, cat, rabbit, ferret, horse, mule, donkey, hamster, or other domesticated pet.

The present disclosure in other perspective refers to the prevention or treatment methods for angiogenic or vascular diseases, and anti-angiogenic methods, including the steps for administering into an individual in need of the antibody or the above composition.

The fusion protein can be provided in a pharmaceutical composition.

The methods of the present disclosure include procedures for administering a pharmaceutical composition of pharmaceutically effective dose for individuals in need of inhibition of angiogenesis. The individual can be a mammal such as dog, cat, ferret, cow, horse, rabbit, mouse, rat, or human, but it is not limited thereto. For example, in one embodiment, the individual can be a chicken, turkey, or other non-mammalian animal. The pharmaceutical composition can be administered via a suitable way including parenterally, subcutaneously, intraperitoneally, intrapulmonarily or intranasally, and if necessary, intralesionally for local treatment. In some embodiments, the dosage of the pharmaceutical composition changes depending on various factors including, but not limited to, the health status and weight of the individual, the severity of the disease, the type of drug, the route and time of administration, and it can be easily determined by those skilled in the art.

In other aspects, the present disclosure refers to the methods of cancer prevention or treatment, including administering procedures of the composition or the antibody to an individual in need of the antibody and the composition or the pharmaceutical composition for cancer prevention or treatment including the antibody.

Cancer is not limited as long as it is treatable with the antibodies of the present disclosure. Specifically, the antibody can prevent the occurrence or progression of cancer by inhibiting angiogenesis. Examples of the cancer include esophageal cancer, stomach cancer, large intestine cancer, rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid cancer blood cancer, but is not limited thereto.

Unless defined otherwise, all technical and scientific terms used herein have the same meaning as those appreciated by those skilled in the field to which the present disclosure pertains.

### EXAMPLES

The following examples are intended merely to illustrate the invention and are not constructed to restrict the invention.

### Example 1. Preparation of Fusion Protein

### A. Construct Design

A fusion protein, IGT-427, was designed wherein that VEGF-A binding regions of a VEGF Receptor 1 (VEGFR1) and a VEGF Receptor 2 (VEGFR2) were fused to the C-terminus of the heavy chain (HC) of an anti-Tie2 antibody. The construct was made into a protein expression vector containing human cytomegalovirus IE1 (CMV-IE1) promoter/enhancer. The amino acid sequences of the heavy chain (HC) and light chain (LC) are shown in Table 1 above.

### B. Expression and Purification of IGT-427

Plasmid DNAs encoding IGT-427 were transiently co-transfected into ExpiCHO-S Cells based on manufacturer's instructions (Gibco). An equal amount of heavy and light chain DNAs was used. Then, the transfected cells were incubated at 32° C. with a humidified atmosphere of 5% CO₂ in air with shaking for Max titer protocol. After 12 days of incubation, the culture medium containing secreted fusion protein was collected, centrifuged to remove the cells, and the culture supernatant was collected and filtered. The fusion protein was purified using AKTAPure purification system (Cytiva) with an affinity column of HiTrap MabSelect Sure Protein A (Cytiva) in binding/wash buffer (20 mM Sodium phosphate, pH 7.2 and 150 mM NaCl) and elution buffer (25 mM Sodium acetate, pH 3.3), thus being immediately neutralized by adding 100 μl of 1 M Tris-HCl, pH 8.8 per milliliter of fraction. The pooled fractions were then buffer exchanged into PBS, pH 7.4 using a centrifugal filter unit (Amicon). The consequent final sample was kept stored until further use. Concurrently, the fusion protein was undergone a quality control studies such as SDS-PAGE (Invitrogen™ Novex™ WedgeWell), SEC-HPLC (Agilent Infinity 1260 and a column of 300 Å pore size) and endotoxin measurement (Charles River Cartridge <0.05EU/ml sensitivity) and Surface Plasmon Resonance (SPR Biacore).

### Example 2. Preparation of Orthologous Tie2 Proteins

### A. Construct Design

The Ig3 and FNIII(1-3) domains of three orthologous proteins of Tie2 (human, rabbit and mouse), were chosen as binding proteins to IGT-427. The domains of each Tie2 orthologues were cloned into pFuse-mouse IgG1 Fc2 vector that is composed of an elongation factor 1-alpha (EF-1α) promotor and an IL2 signal peptide, along with a serial C-terminal hexa-Histidine and thrombin protease cleavage site (LVPRGS) between Tie2 and the IgG1 Fc sequences. Sequences of human (SEQ ID NO:2), rabbit (SEQ ID NO:3), and mouse (SEQ ID NO: 4) Tie2 orthologues are shown in Table 1 above.

### B. Expression and Purification of Tie2 Orthologues

To produce Tie2 orthologues, Expi293F (Gibco) cells capable of producing recombinant proteins with high efficiency were used. The orthologous Tie2 proteins were transiently expressed in Expi293F cell line by referring to manufacturer's instruction manual (Gibco). Post 4 days of transfection and incubation at 37° C. with a humidified atmosphere of 8% CO₂ in air with shaking, the resulting culture medium was collected and centrifuged to remove the cells. The culture supernatant containing secreted antibodies was isolated and stored at 4° C. or immediately purified using an AKTAPure purification device (Cytiva) equipped with an affinity column (HiTrap MabSelect Sure Protein A, Cytiva) in two different buffer systems of binding and wash (20 mM Sodium phosphate, pH 7.2 and 150 mM NaCl) and elution buffer (25 mM Sodium acetate, pH 3.3). The Tie2 proteins were immediately neutralized by adding 100 μl of 1 M Tris-HCl, pH 8.8 per milliliter of fraction. The fractions were pooled and then buffer exchanged into PBS, pH 7.4 through a protein centrifugal filter (Amicon) and stored at −80° C. As a following process, the proteins were analyzed for a quality control studies such as SDS-PAGE (Invitrogen™ Novex™ WedgeWell), SEC-HPLC (Agilent Infinity 1260 and a column of 300 Å pore size) and Surface Plasmon Resonance (SPR).

### Example 3. Affinity Measurements of Fusion Protein (IGT-427) Against Tie2 Orthologues

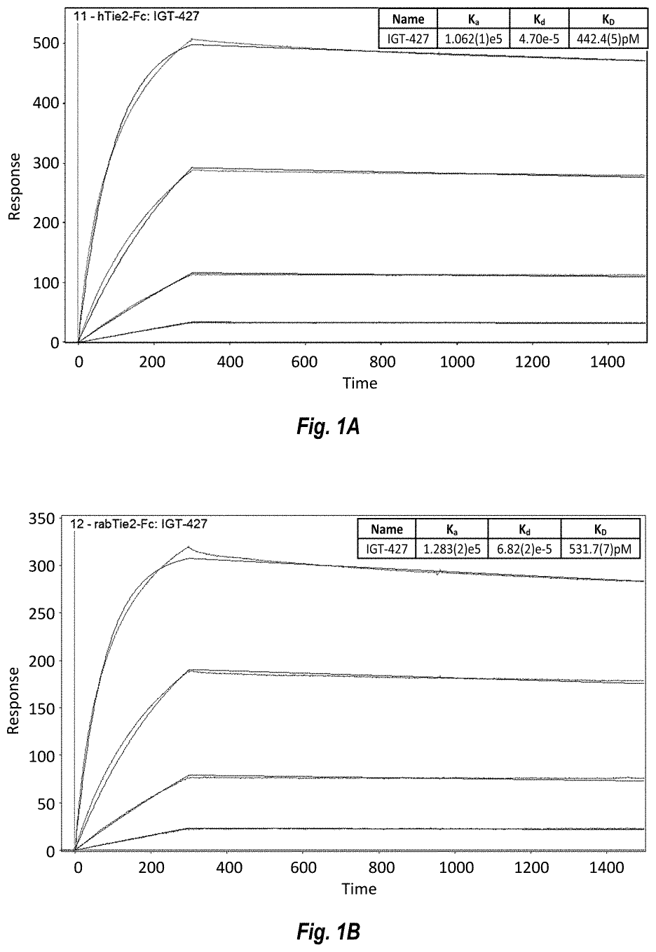

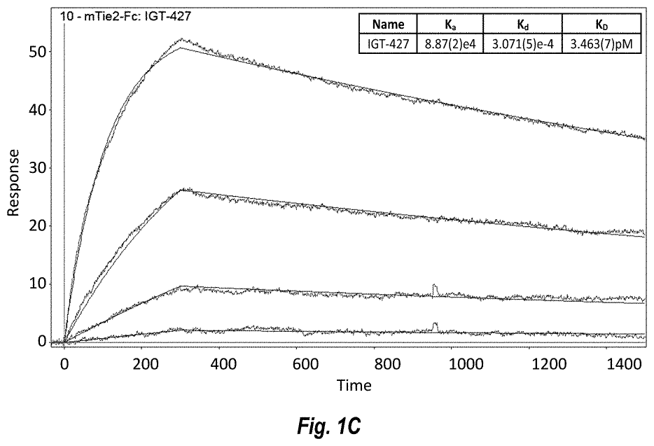

The affinities of the fusion protein against orthologous Tie2 proteins were measured by SPR system (Biacore 3000). CM5 SPR chips (Cytiva) were modified with either monomeric or dimeric forms of human, rabbit, or mouse Tie2 using EDC/NHS chemistry (where the dimeric forms were Fc fusions). The sensor chip was activated with EDC (0.2 M) and NHS (0.05 M) for 7 minutes, followed by injection of the Tie2 constructs at 2.5 μg mL⁻¹ in 10 mM sodium acetate at pH 5 to reach an antigen surface density of approximately 200 RU. The surfaces were then deactivated with a 7 min injection of 1 M ethanolamine·HCl pH 8.5. Activation and deactivation reagents were purchased from Cytiva. After immobilization of the Tie2 constructs, the surfaces were conditioned with two 5 s pulses of 300 mM phosphoric acid. Fusion protein in running buffer (10 mM HEPES pH 7.4, 150 mM NaCl, 3 mM EDTA, 2 mg/mL BSA, and 0.05% polysorbate-20) was injected at 100 nM, 33.3 nM, 11.1 nM, 3.7 nM, and 1.2 nM over these immobilized Tie2 surfaces at 30 μL min-1 for 5 min, and the dissociation was monitored for 20 min Surface regeneration was performed with 10 s injections of 300 mM phosphoric acid. Sensorgrams were fit to a 1:1 Langmuir binding model using Scrubber software (BioLogic). The K_{D} values and SPR sensorgrams were shown in the following Table 2 and FIGS. 1A-1C, respectively.

TABLE 2
Affinities of fusion protein IGT-427 to
antigen Tie2-Ig3-FNIII(1-3) orthologues

| Antigen | Kₒₙ (M⁻¹ s⁻¹) | K_{off} (1/s) | K_{D} (M) |
|---|---|---|---|
| Human dimericTie2 | 1.06E+05 | 4.70E−05 | 4.42E−10 |
| Rabbit dimericTie2 | 1.28E+05 | 6.82E−05 | 5.32E−10 |
| Mouse dimericTie2 | 8.87E+04 | 3.07E−04 | 3.46E−09 |
| Human monomeric Tie2 | 1.59E+05 | 4.00E−05 | 2.50E−10 |
| Rabbit monomeric Tie2 | 1.65E+05 | 6.49E−05 | 3.93E−10 |
| Mouse monomeric Tie2 | 1.19E+04 | 4.33E−04 | 3.62E−09 |

### Example 4. Inhibition of VEGFR2 Phosphorylation by IGT-427

HUVECs (1×10⁵ cells/ml) were cultured in EGM-2 medium (Lonza) at 37° C. in a 60 mm culture dish. Cells at 90% confluency were incubated with supplement-free EBM-2 medium for 4 hours for starvation. The starved HUVECs were pre-treated with an IGT-427 or EYLEA® at the indicated concentration for 30 min and then treated with recombinant human VEGF for 2 min. The cells were washed with cold PBS, treated with lysis buffer (10 mM Tris-Cl pH 7.4, 150 mM NaCl, 5 mM EDTA, 10% glycerol, 1% Triton X-100, protease inhibitor, phosphatase inhibitor), and lysed at 4° C. for 20 min. Then, the cell lysates were prepared by centrifugation at 13000 rpm for 15 min Protein concentration in the supernatant was quantitated by BCA assay. By adding 4×SDS sample buffer, cell lysates were prepared and the cell lysates were subjected to SDS PAGE and proteins were transferred to a nitrocellulose membrane (GE).

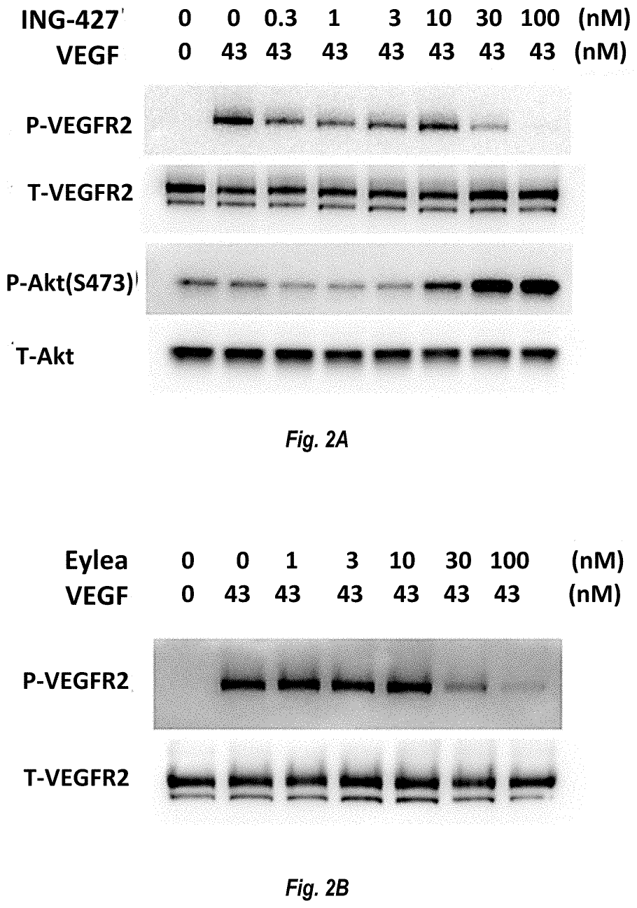

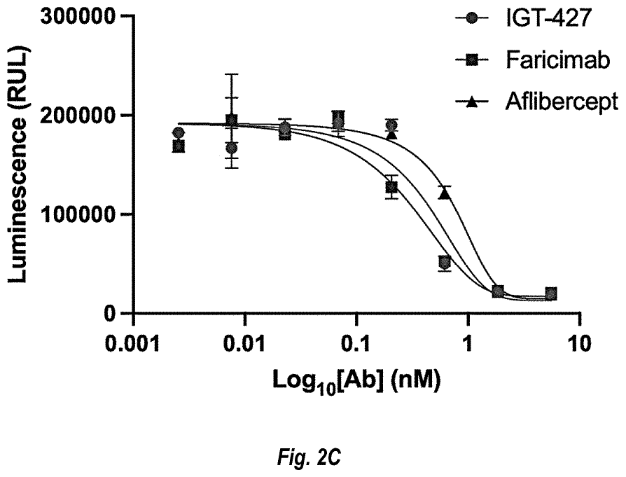

To investigate VEGFR2 and Akt phosphorylation, the blot was blocked with 5% skim milk-containing TBS-T at room temperature (RT) for 1 hour, and incubated with anti-phospho-VEGFR2 antibody (Tyr1175) or anti-phospho Akt (S473) antibody at 4° C. for about 16 hours. The signals of phospho-VEGFR2 (Tyr1175) or pAkt (S473) were visualized by an enhanced chemiluminescence (ECL). Then, the membrane was incubated in a stripping buffer (Thermo) for 15 minutes, and then re-probed with an anti-VEGFR2 or anti-Akt antibody to determine the amount of total VEGFR2 and total Akt. VEGF-induced VEGFR2 phosphorylation at Tyr1175 was inhibited by IGT-427 in a concentration dependent manner and the inhibition is comparable to one by EYLEA® (FIGS. 2A and 2B). In addition, IGT-427 induced the phosphorylation of Akt in a dose-dependent manner.

VEGF reporter assay was performed using VEGF reporter bioassay kit (PROMEGA™). Briefly, 0.4 ml of KDR/NFAT-RE HEK293 cells were added in 4.6 ml of DMEM medium containing 10% FBS (assay buffer) and 25 μl of cell suspension were plated in a white, flat-bottom 96-well assay plate (Corning). IGT-427, faricimab or aflibercept was added at the concentration of 3× higher concentration of 50 nM and threefold serial dilutions in 25 μl of assay buffer, and recombinant human VEGF of 3×higher concentration of 40 ng/ml in 25 μl of assay buffer was added in the plates. After 6 hours of incubation at 37° C., 75 μl of BIO-GLO™ reagent provided in the kit were added, and luminescence was measured using the GloMax® Discovery System (PROMEGA™). The IC 50 values of IGT-427, faricimab or aflibercept were approximately 0.48 nM, 0.32 nM, or 0.75 nM, respectively (FIG. 2C), suggesting that VEGF inhibition by IGT-427 is comparable to that by either faricimab or aflibercept.

### Example 5. Stronger and More Persistent Activation of Tie2 by IGT-427

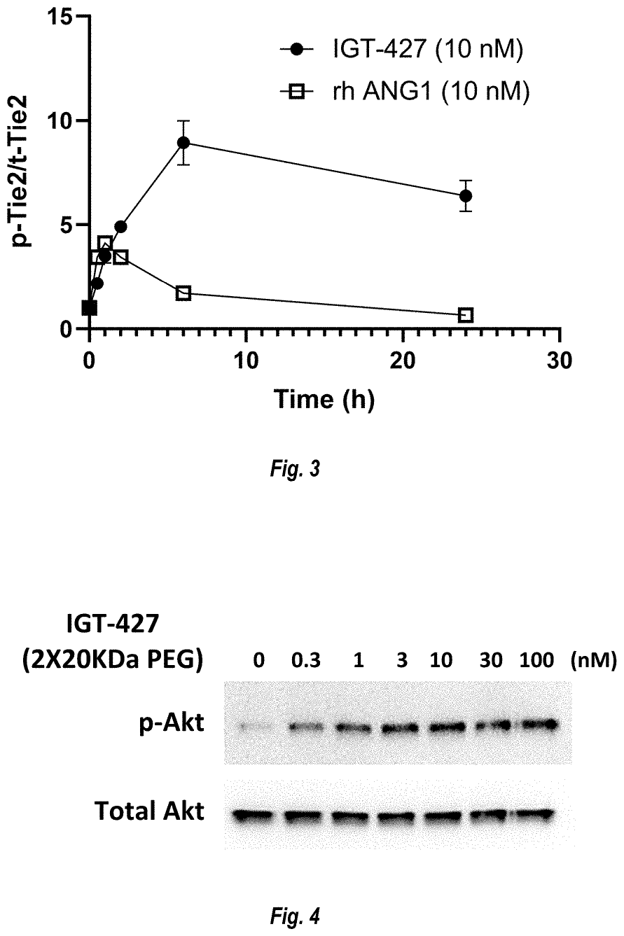

CHO cells overexpressing full-length of human Tie2 (CHO-hTie2, 1×105 cells/ml) were cultured in DMDM medium (Thermo Fisher) at 37° C. in a 60 mm culture dish. Cells at 90% confluency were incubated with supplement-free DMEM medium for 4 hours for starvation. The starved CHO cells were treated with an IGT-427 or recombinant human angiopoietin-1 (R&D Systems®) at a concentration of 10 nM for various duration (30 min, 1 hour, 2 hours, 6 hours, and 24 hours). The cells were washed with cold PBS, treated with lysis buffer (10 mM Tris-Cl pH 7.4, 150 mM NaCl, 5 mM EDTA, 10% glycerol, 1% Triton X-100, protease inhibitor, phosphatase inhibitor), and lysed at 4° C. for 20 min. Then, the cell lysates were prepared by centrifugation at 13000 rpm for 15 min. The lysates were then used to detect the levels of phospho-Tie2 and total Tie2 signals using pTie2 and total Tie2 ELISA assay kits (R&D Systems®). The phospho-Tie2 signals were normalized by the total Tie2 signals. IGT-427 showed stronger and more persistent pTie2 signal, compared with angiopoietin-1 (FIG. 3).

### Example 6. Dose-Dependent Activation of Akt Signaling by IGT-427

HUVECs (1×105 cells/ml) were cultured in EGM-2 medium (Lonza) at 37° C. in a 60 mm culture dish. Cells at 90% confluency were incubated with supplement-free EBM-2 medium for 4 hours for starvation. The starved HUVECs were treated with various concentrations of IGT-427 (0.3, 1, 3, 10, 30, 100 nM) for 30 min. The cells were washed with cold PBS, treated with lysis buffer (10 mM Tris-Cl pH 7.4, 150 mM NaCl, 5 mM EDTA, 10% glycerol, 1% Triton X-100, protease inhibitor, phosphatase inhibitor), and lysed at 4° C. for 20 min. Then, the cell lysates were prepared by centrifugation at 13000 rpm for 15 min Protein concentration was quantitated by BCA assay. By adding 4×SDS sample buffer, cell lysates were prepared and the cell lysates were subjected to SDS PAGE and proteins were transferred to a nitrocellulose membrane (GE). To investigate Akt phosphorylation, the blot was blocked with 5% skim milk-containing TBS-T at room temperature (RT) for 1 hour, and incubated with anti-phospho Akt (S473) antibody at 4° C. for about 16 hours. The signals of pAkt (S473) were visualized by an enhanced chemiluminescence (ECL). Then, the membrane was incubated in a stripping buffer (Thermo) for 15 minutes, and then re-probed with an anti-Akt antibody to determine the amount of total Akt. As shown in FIG. 4, there was a dose-dependent, gradual increase of phospho-Akt signaling by IGT-427.

### Example 7. Bypassing Ang2 Signaling by IGT-427

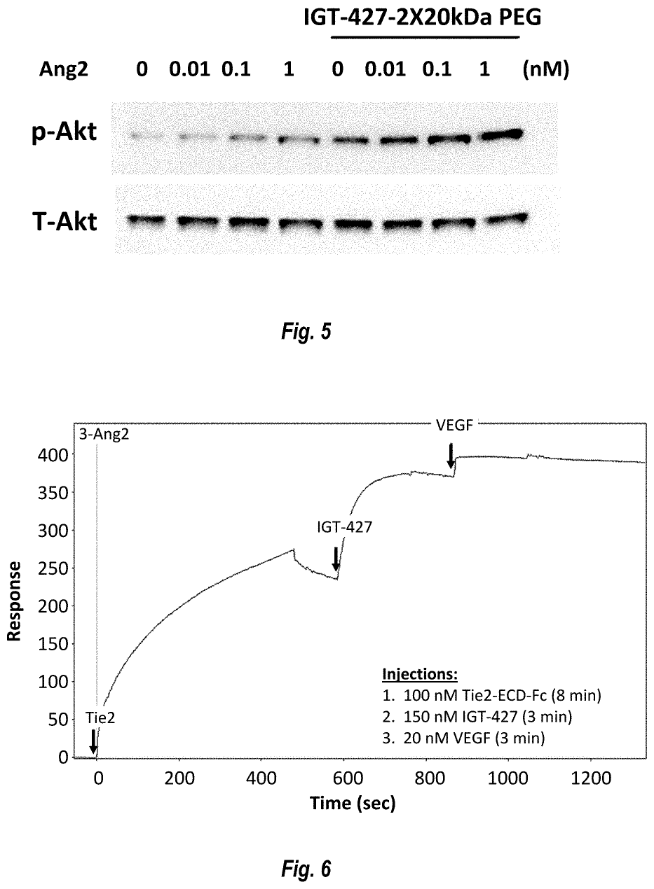

HUVECs (1×105 cells/ml) were cultured in EGM-2 medium (Lonza) at 37° C. in a 60 mm culture dish. Cells at 90% confluency were incubated with supplement-free EBM-2 medium for 4 hours for starvation. The starved HUVECs were pre-treated with recombinant human Angiopoietin-2 (R&D Systems®) at a concentration of 0.01, 0.1, 1 nM for 30 min and then treated with 2×20 kDa pegylated IGT-427 at a concentration of 30 nM for 60 min. The cells were washed with cold PBS, treated with lysis buffer (10 mM Tris-Cl pH 7.4, 150 mM NaCl, 5 mM EDTA, 10% glycerol, 1% Triton X-100, protease inhibitor, phosphatase inhibitor), and lysed at 4° C. for 20 min. Then, the cell lysates were prepared by centrifugation at 13000 rpm for 15 min Protein concentration was quantitated by BCA assay. By adding 4× SDS sample buffer, cell lysates were prepared and the cell lysates were subjected to SDS PAGE and proteins were transferred to a nitrocellulose membrane (GE). To investigate Akt phosphorylation, the blot was blocked with 5% skim milk-containing TBS-T at room temperature (RT) for 1 hour, and incubated with anti-phospho Akt (S473) antibody at 4° C. for about 16 hours. The signals of pAkt (S473) were visualized by an enhanced chemiluminescence (ECL). Then, the membrane was incubated in a stripping buffer (Thermo) for 15 minutes, and then re-probed with an anti-Akt antibody to determine the amount of total Akt. Recombinant Angiopoientin-2 (Ang2) induced a weak phosphorylation of Akt in a concentration-dependent manner. However, IGT-427 increased the Akt phosphorylation further in the presence of the recombinant Angiopoientin-2, demonstrating that IGT-427 overcomes Ang2 signaling (FIG. 5).

### Example 8. Dual Binding Capability of IGT-427

The dual binding characteristics of IGT-427 to its antigens, Tie2 and VEGF, were examined by surface plasmon resonance (SPR) system (BIACORE™ 3000). Recombinant human full-length Ang2 (R&D Systems®) was captured on CM5 SPR chips (Cytiva) and human dimeric form of Tie2 (100 nM) was injected first, followed by the sequential injections of IGT-427 (150 nM) and human VEGF (20 nM) (R&D Systems®). The sensorgram showed the sequential increases of the signals, demonstrating that IGT-427 is able to bind to the Ang2-Tie2 complex and VEGF simultaneously (FIG. 6).

### Example 9. Inhibition of TNF-Alpha-Induced Apoptosis by IGT-427

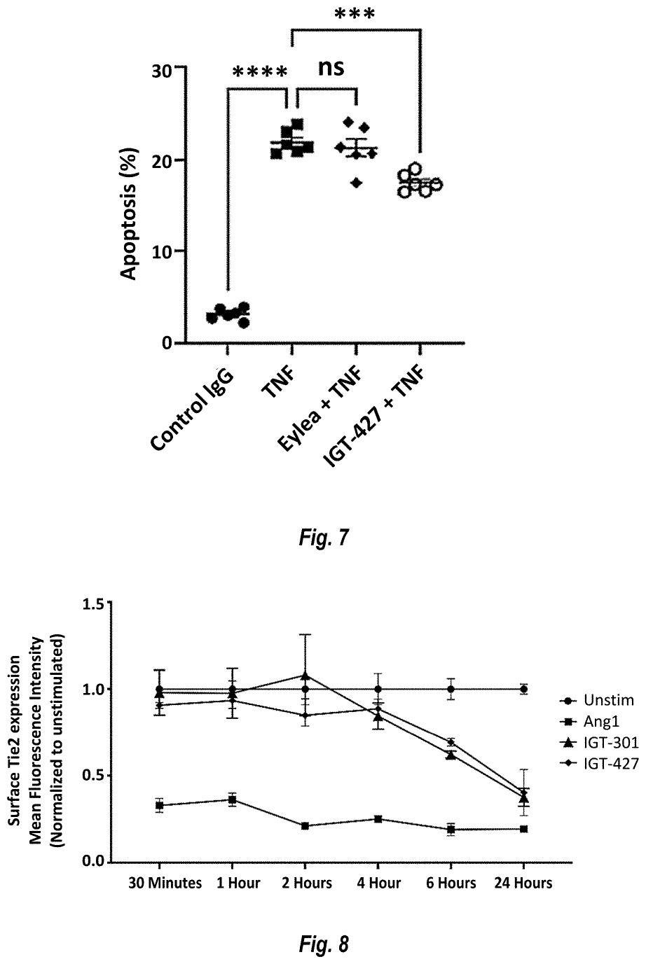

HUVECs (1×10 5 cells/ml) were cultured in EGM-2 medium (Lonza) at 37° C. in a 60 mm culture dish. Cells at 90% confluency were pre-treated with an IGT-427 or EYLEA® for 60 minutes and then treated with recombinant human TNF-alpha (50 ng/ml) for 24 hr. Apoptotic cells were stained by APO-BrdU™ TUNEL Assay Kit (Thermo Fisher, A23210), and determined by Attune (Thermo Fisher). Briefly, cells were suspended in 0.5 mL of PBS and 5 mL of 1% (w/v) paraformaldehyde in PBS was added to the cell suspension and then cell suspension was placed on ice for 15 minutes. Cells were centrifuged for 5 minutes at 300×g and were washed twice in 5 mL of PBS. Cells were resuspended in 0.5 mL of PBS and 5 mL of ice cold 70% (v/v) ethanol was added to the cell suspension and cell suspension was placed for a minimum of 30 minutes in a −20° C. freezer. To remove the 70% (v/v) ethanol, cell suspension was centrifuged at 300×g for 5 minutes and then cell pellets were resuspended with 1 mL of wash buffer provided in the kit. Cell pellet was incubated in 50 μL of the DNA labeling solution (10 μL of reaction buffer, 0.75 μL of TdT enzyme, 8.0 μL of BrdUTP and 31.25 μL of dH2O) for 60 minutes at 37° C. At the end of the incubation time, 1.0 mL of rinse buffer was added to the cells and the cells were centrifuged at 300×g for 5 minutes. After another wash with rinse buffer, cells were incubated in 100 μL of antibody staining solution (5.0 μL of the Alexa Fluor™ 488 dye—labeled anti BrdU antibody) with 95 μL of rinse buffer) for 30 minutes at room temperature. Apoptotic cells were analyzed by Attune flow cytometry (Thermo Fisher). TNF-alpha induced apoptosis of HUVEC cells and IGT-427 inhibited significantly TNF-alpha-induced apoptosis, but EYLEA® did not (FIG. 7).

### Example 10. Measurement of Surface Tie2 Level

In preparation for the assay, CHO-hTie2 cells were grown to confluency on a 10 cm tissue culture dish. CHO-hTie2 cells were washed with Dulbecco's phosphate-buffered saline (DPBS), treated with trypsin to detach cells, counted, centrifuged for 5 minutes at 200 g, and resuspended at a density of 0.05×106 cells per mL. To seed the assay plates, 1 mL of the cell suspension was aliquoted to each well of a 24-well tissue culture plate and the plates incubated overnight at 370° C. On the following day, the media was removed from the tissue culture plates and replaced with 0.5 mL of either DMEM alone for unstimulated groups or DMEM containing 10 nM Angiopoietin 1 (R&D Systems® #923-AN), 10 nM IGT-301 (ITP-006), or 10 nM IGT-427 (ITP-016). The assay plates were returned to the 370° C. tissue culture incubator for the pre-determined length of time (0.5 hours, 1 hour, 2 hours, 4 hours, 6 hours, or 24 hours).

Following incubation, the tissue culture plates were placed on ice, washed with cold DPBS, and detached by adding 0.5 mL of a non-enzymatic cell dissociation buffer (PeproTech® #CPD-125). Cells were incubated on ice for 5 minutes and collected by adding 1 mL of FACS Buffer (0.5% BSA in DPBS) and pipetting gently to detach cells still adhered to the culture surface. The resulting cell suspension was collected into 5 mL Falcon FACS tubes, centrifuged for 5 minutes at 300 g to pellet the cells, and the supernatant was aspirated. Cells were washed once with 1 mL of FACS Buffer and the supernatant aspirated leaving ˜100 μL of residual FACS Buffer in the tube. Cells were then stained by adding 100 μL of a staining cocktail consisting of 99.5 μL FACS Buffer and 0.5 μL of PE-Tie2 (BioLegend® #334205). Samples were covered with parafilm and incubated at 40° C. in the dark for 30 minutes.

After incubation, cells were washed twice with 1 mL of FACS Buffer as before and were then either resuspended in a final volume of 400 μL FACS Buffer and analyzed immediately or fixed for analysis the following day. For fixation, cells were washed twice as above, resuspended in 200 μL 4% paraformaldehyde for 30 minutes at 40° C. in the dark, washed once with 1 mL FACS buffer, resuspended at a final volume of 400 μL FACS Buffer, and stored covered at 40° C. overnight. Samples were acquired using the Attune NXT Acoustic flow cytometer (Thermo Fisher) and files analyzed using FlowJo™ (Tree Star, Inc.). IGT-427 treatment resulted in slower disappearance and more presence of surface Tie2, compared to the endogenous Tie2 agonist, Angiopoietin-1 (Ang1) (FIG. 8).

### Example 11. Blocking Tie2 Cleavage

Tie2 is known to be cleaved and downregulated in human, mouse and endothelial cells in various inflammatory conditions, leading to the increased levels of soluble Tie2 (sTie2) (Thamm et al., Critical Care Medicine, 2018. 46:e928-e936). Blocking global matrix metalloprotease (MMP) is known to be sufficient to prevent Tie2 cleavage and vascular leakage (Thamm et al., Critical Care Medicine, 2018. 46:e928-e936; Sung et al., 2011. The Journal of Clinical Endocrinology & Metabolism 96:E1148-E1152; Findley et al., 2007, Arteriosclerosis, Thrombosis, and Vascular Biology 27:2619-2626). MMP-14 was implicated as being the principal Tie2 cleavage and Tie2 is cleaved at multiple sites within fibronectin type 3 domains by matrix metalloprotease-14 (Idowu, T O et al., eLife, Aug. 24, 2020, 9:e59520).

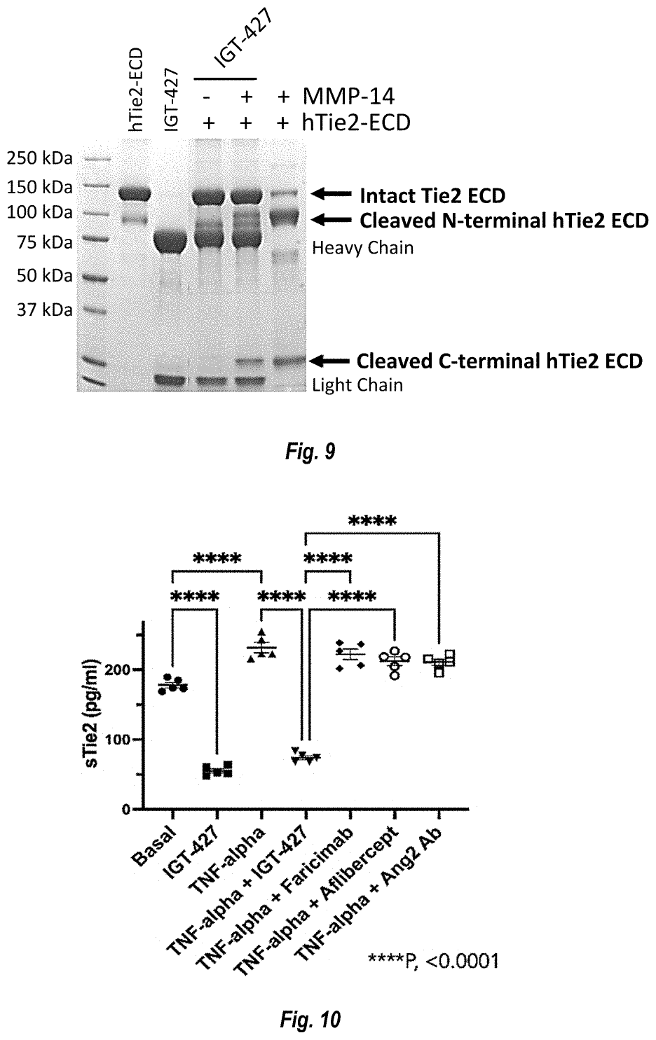

Recombinant human MMP-14 (R&D Systems®, Cat. #, 918-MP-010, 0.5 ug) was mixed with the dimeric human Tie2 extracellular domain (ECD)-human IgG fusion protein (hTie2-ECD, 5 ug) either in the absence or presence of IGT-427 (same molar concentration of hTie2-ECD). The mixture was incubated at 30° C. for 16 hours and subject to SDS-PAGE (reducing condition). Consistent with the previous report, co-incubation of MMP-14 and human Tie2 resulted in significant reduction of intact hTie2-ECD, with the appearance of shorter, cleaved fragments. However, IGT-427 blocked significantly the cleavage and reduction of intact human Tie2-ECD protein by MMP-14 (FIG. 9).

Without wishing to be bound by theory, and based on this evidence, it is believed that the bispecific comprising the anti-Tie2 antibody or antigen-binding fragment which binds to Tie2 Ig3-FNIII (1-3) domain comprising SEQ ID NO: 2, 3, or 4, or more specifically the amino acid sequence of SEQ ID NO: 20 and/or the amino acid sequence of SEQ ID NO: 21, and the VEGF binding domain will reduce the shedding of Tie2 by combining the effect of blocking the binding of MMP-14 to human Tie2 with the effect of inhibiting the cleavage of human Tie2-ECD protein caused by VEGF. This provides a particularly beneficial result of preserving Tie2 at the cell surface membrane.

### Example 12. Soluble Tie2 (sTie2) Measurement

HUVECs (1×105 cells/ml) were cultured in EGM-2 medium (Lonza) at 37° C. in a 60 mm culture dish. Cells at 90% confluency were pre-treated with an IGT-427, Faricimab, or recombinant human angiopoietin-1 (R&D Systems®) at a concentration of 10 nM for 60 minutes, and then treated with recombinant human TNF-alpha (50 ng/ml) for 24 hr. The cell supernatant was collected and centrifugated at 13000 rpm for 10 min Soluble Tie2 in the cell supernatant was measured by total Tie2 ELISA assay kits (R&D Systems® IGT-427 suppressed soluble Tie2 level both in basal and in TNF-alpha-induced conditions, while other agents did not (FIG. 10).

### Example 13. TEER (Trans-Endothelial Electrical Resistance) Assay

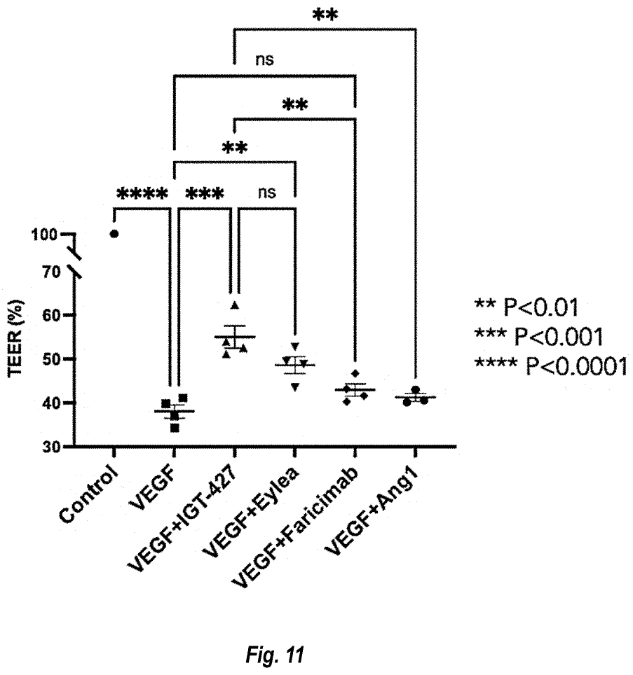

HUVECs (2×105 cells) were cultured in a cell culture insert in 24 well plate (Corning) in EGM-2 medium (Lonza) at 37° C. After two days of incubation, medium was changed to EGM-2 medium containing 0.5% FBS. After the following two days of incubation, a cell culture insert was placed into CellZscope® (NanoAnalytics) and TEER (Trans-endothelial electrical resistance) was measured continuously in a CO2 incubator at 37° C. After confirming the cell barrier formation, recombinant human VEGF (R&D) at a concentration of 10 ng/ml was added in the lower compartment. Various agents, such as IGT-427, Faricimab, Aflibercept, or recombinant human Angiopoietin-1 (R&D) at a concentration of 10 nM, were added into the lower compartment and TEER was monitored for the following 48 hours post VEGF treatment. VEGF disrupts endothelial barrier integrity and IGT-427 was better than faricimab and aflibercept, and Ang1 in recovering endothelial barrier integrity from VEGF-induced damage (FIG. 11).

### Example 14. Suppression of CNV (Choroidal Neo-Vascularization) by Intravitreally Injected IGT-427

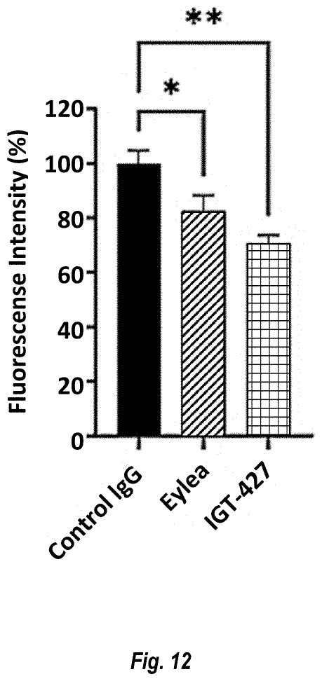

Chinchilla rabbits (male, 2.0˜2.5 kg) were anesthetized by applying eye drops (Mydriacyl ophthalmic solution, 1%) to the right eyeball, and the right eyeball was irradiated by laser (Elite, Lumenis®, USA) at 532 nm, power 150 mW, duration 0.1 sec, to generate six spots around the optic nerve. IGT-427, EYLEA® or control IgG (50 μl injection volume/eye, EYLEA® (800 μg), IGT-427 (885 μg), control IgG (716 μg)) was intravitreally administered at the date of Choroidal Neo-Vascularization (CNV) induction using a syringe equipped with a 3 gauge needle in the right eye of the anesthetized animal. The molar ratio of EYLEA®, IGT-427 & control IgG is 1:0.65:0.68. Different moles were used due to some restrictions on the concentrations and volumes. On Day 0, 7, and 14, the animals were anesthetized by applying eye drops (Mydriacyl ophthalmic solution, 1%) to the right eyeball, and 1 mL of fluorescein sodium salt solution (2%) was injected intravenously to take the fundus images using fundus camera (TRC-50IX, TOPCON, Japan) within 2 minutes. Evaluation of the retinal CNV area and efficacy was performed using retinal fluorescein fundus photography and the Image analysis was performed using ImageJ software (NIH, Bethesda, Md.) to verify the fluorescence intensity of the CNV lesion site. IGT-427 suppressed retina leakage by 30%, compared to control IgG. In contrast, EYLEA® was able to inhibit retina leakage by 20% (FIG. 12).

### Example 15. IGT-427 Variants for PEGylation

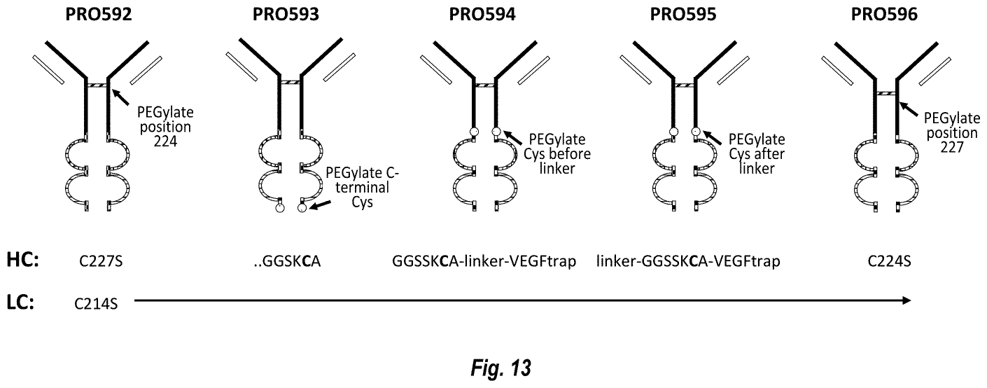

To extend the ocular half-life of IGT-427, the heavy and light chain plasmids of IGT-427 were modified to make five different antibody constructs for PEGylation (outlined in FIG. 13). All five variants have a light chain with the mutation C214S (EU numbering), which, in combination with the C218S mutation (EU numbering) on each heavy chain, removes the interchain disulfide bond between heavy and light chains. In addition, one or both heavy chain cysteines in the hinge region of IGT-427 was replaced with serine leaving either one or zero of the interchain disulfide bonds between heavy chains. In the case of PRO592 and PRO596, a single disulfide bond remains to be reduced and PEGylated. In the case of PRO593, PRO594, and PRO595, all interchain disulfide bonds are removed and a new cysteine residue is introduced for PEGylation. PRO593 has a cysteine introduced on the C-terminus of the heavy chain, whereas PRO594 and PRO595 each have a cysteine introduced on the linker domain between the Fc domain and the VEGF trap domain. Together these variants represent a set of constructs with diverse PEGylation sites that were tested for PEGylation efficiency, antigen binding, and bioactivity. Each IGT-427 variant was expressed using the Expi293 transient expression system. Clarified cell culture supernatants were purified with a HiTrap mAbselect PrismA column (Cytiva). Eluted antibody was dialyzed into 1×PBS and concentrated to 10 mg/mL for PEGylation studies.

### Example 16. PEGylation Reaction Screening

Each of the IGT-427 variants at 10 mg/mL were reduced with 10 or 20 mM cysteamine for one hour at room temperature and then buffer exchanged into PEGylation buffer (50 mM sodium phosphate, 150 mM NaCl, 2.5 mM EDTA, pH 7.2). Ten molar equivalents of 20 kDa linear PEG-maleimide was added to the buffer exchanged antibodies, and the PEGylation reactions were allowed to proceed at room temperature for 1.5 hours. The reaction mixtures were analyzed by non-reduced SDS-PAGE, which revealed that the variants showed at most ˜50% conversion to doubly PEGylated species.

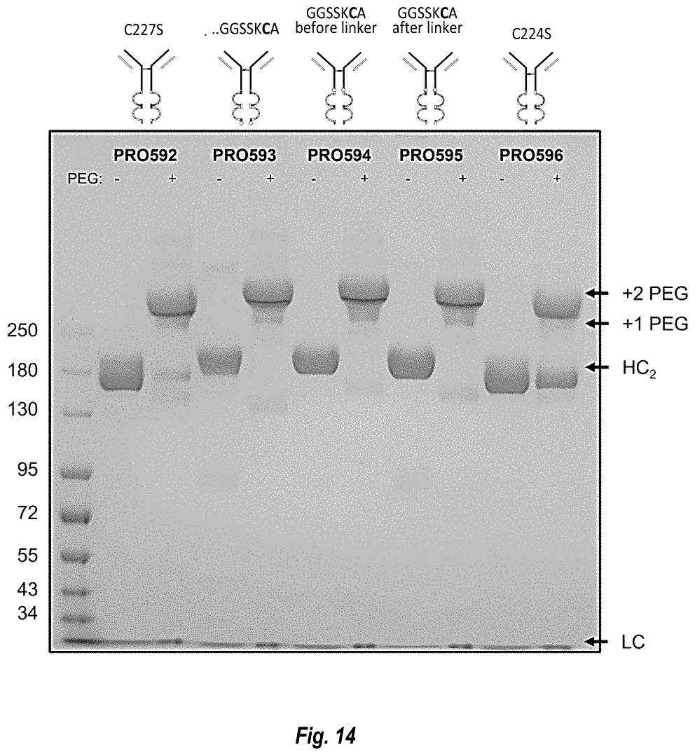

To further optimize the PEGylation efficiency, reduction conditions were screened using PRO593, the variant that showed the best conversion under mild reducing conditions. PRO593 at 10 mg/mL was reduced with either 10 or 100 mM cysteamine for 1 hour, 1 or 10 mM DTT for 15 minutes, or 2- or 20-fold molar excess of TCEP (tris(2-carboxyethyl)phosphine) for 15 minutes. After each reduction, PRO593 was buffer exchanged into PEGylation buffer and PEGylated with 10-fold molar excess of 20 kDa linear PEG-maleimide for 1.5 hours. From this set of reduction conditions, it was observed that 20-fold molar excess of TCEP resulted in almost complete conversion of PRO593 to a species with two PEG additions. All five of the IGT-427 variants were then reduced with 20-fold molar excess of TCEP at room temperature for 15 minutes, followed by buffer exchange into the PEGylation buffer and PEGylation with 10-fold molar excess of 20 kDa linear PEG-maleimide. The results of the optimized reaction conditions are shown in FIG. 14. Antibodies with two PEG additions were purified from the reaction mixture using a HiTrap SP-HP cation exchange chromatography column (Cytiva) and gradient salt elution. PEGylated antibodies were dialyzed into 1×PBS and concentrated to 10 mg/mL, formulated with 0.01% polysorbate-20, and filtered through a 0.2 μm filter.

### Example 17. PEGylated IGT-427 Variant Characterization

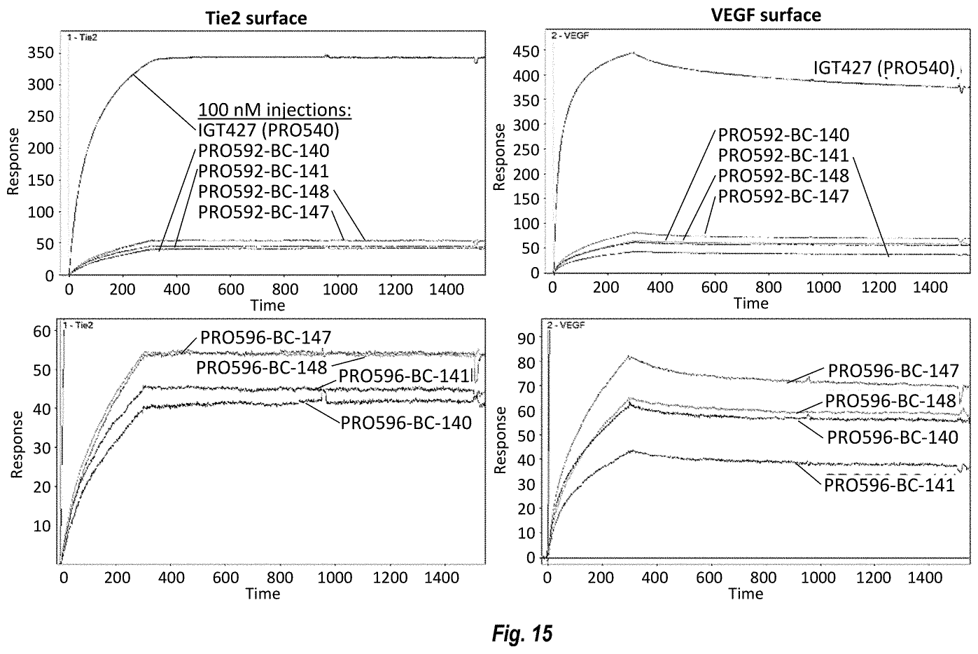

Binding Tie2 or VEGF was characterized by SPR (FIG. 15). A 100 nM solution of each antibody was injected at 30 μL/min for 300 seconds over a surface containing immobilized Tie2 or VEGF. Relative binding signals after 300 seconds were used to determine differences in binding.

### Example 18. Ocular PK Study

To assess the effect of PEGylation and PEG molecular weight on IGT-427 ocular pharmacokinetics, 500 μg intravitreal injections of EYLEA®, faricimab, IGT-427, (2×20 kDa PEG)-IGT-427, and (2×40 kDa PEG)-IGT-427 were administered to male New Zealand White rabbits. For each test article group, three or four animals were dosed at day 0. On days 1, 3, 7, and 14 post-dose, blood was collected from either three or four animals that were then euthanized by intravenous barbiturate overdose prior to both eyes being harvested, snap frozen in liquid nitrogen and dissected for the collection of aqueous humor, vitreous humor, retina, and choroid. Vitreous humor samples were diluted 1:5 in 1× PBST without homogenization and stored at −70° C. until further analysis.

ZALTRAP® was purchased from a commercial source, dialyzed against the EYLEA® formulation buffer (10 mM sodium phosphate, 40 mM NaCl, 0.03% polysorbate-20, 5% sucrose), and diluted to 10 mg/mL in the same buffer.

Faricimab was expressed using the Expi293 transient expression system (ThermoFisher). Clarified cell culture supernatant was purified with a HiTrap MabSelect PrismA column (Cytiva) with 2% ethanol wash to remove endotoxin, followed by a pH gradient elution to remove incompletely assembled antibody. Eluted antibody was dialyzed into 1× PBS, concentrated to 10 mg/mL, formulated with 0.01% polysorbate-20, and filtered through a 0.2 μm filter.

IGT-427 was expressed using the Expi293 transient expression system. Clarified cell culture supernatant was purified with a HiTrap MabSelect PrismA column (Cytiva) with 2% ethanol wash to remove endotoxin. Eluted antibody was dialyzed into 1× PBS, concentrated to 10 mg/mL, formulated with 0.01% polysorbate-20, and filtered through a 0.2 μm filter.

PEGylated IGT-427. PRO593 was expressed using the Expi293 transient expression system (ThermoFisher). Clarified cell culture supernatant was purified with a HiTrap MabSelect PrismA column (Cytiva) with 2% ethanol wash to remove endotoxin. Eluted antibody was dialyzed into 1× PBS and concentrated to 10 mg/mL for PEGylation. Concentrated antibody was reduced with 20-fold molar excess of TCEP, then desalted into PEGylation buffer and incubated with 10-fold molar excess of either 20 kDa linear or 40 kDa branched PEG maleimide for 1.5 hours at room temperature. The PEGylation reaction mixture was purified using a HiTrap SP-HP column (Cytiva) at pH 4.6 with a NaCl gradient. PEGylated antibody in the elution was dialyzed into PBS, concentrated to 10 mg/mL, formulated with 0.01% polysorbate-20, and filtered through a 0.2 μm filter.

### Example 19. Characterization of Samples for PK Study

Non-reduced SDS-PAGE analysis of each test article was performed by mixing 5 μg of protein with 4× LDS (lithium dodecyl sulfate) and running the samples without heating on a 4-12% bis-tris gel at 150V for 60 minutes, followed by staining with SafeStain (ThermoFisher). SEC-HPLC analysis was performed by loading 5 μg of each test article on a Zenix® size exclusion column (SEC)-300 at 0.5 mL/min in 100 mM arginine, 1× PBS, pH 6.7. Binding to rabbit Tie2 and VEGF was characterized by SPR.

SPR was used to assess the binding of the PEGylated IGT-427 variants to either Tie2 or VEGF. FIG. 3 shows the resulting sensorgrams from 100 nM injections of the variants over human Tie2 or VEGF surfaces. Relative to unmodified IGT-427, there is a drastic reduction in the binding signal due to the presence of PEG. However, the relative binding signal of each variant to the rest can be used as a measure of relative affinity for the antigen. While there are slight differences in binding signal for each of the variants, overall, the binding to either Tie2 or VEGF is largely independent of the PEGylation site.

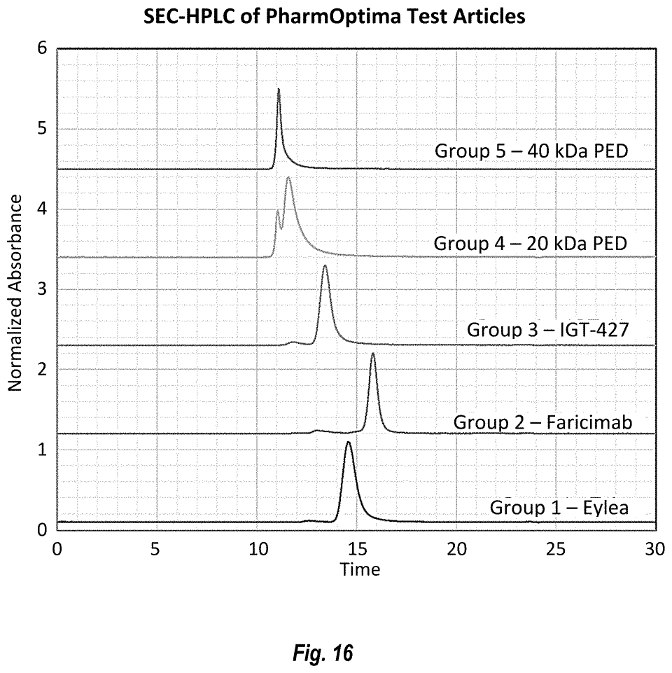

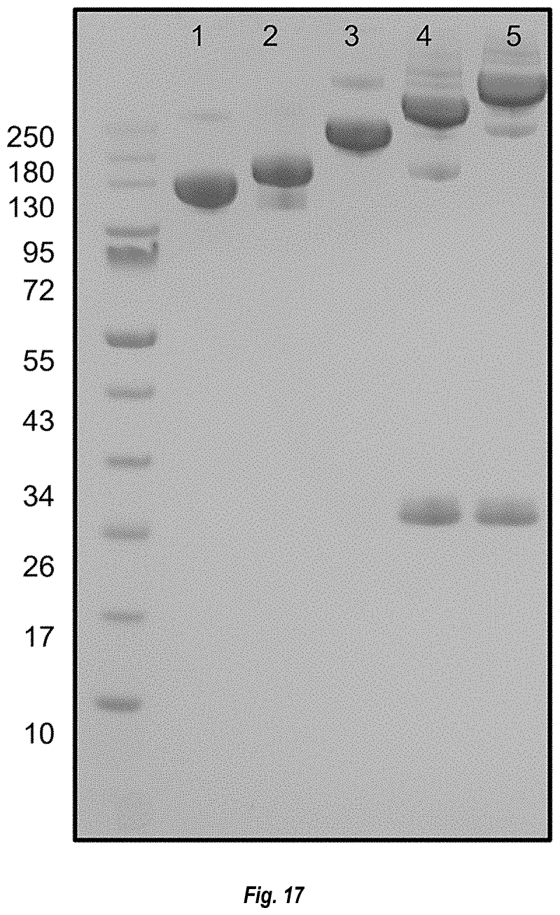

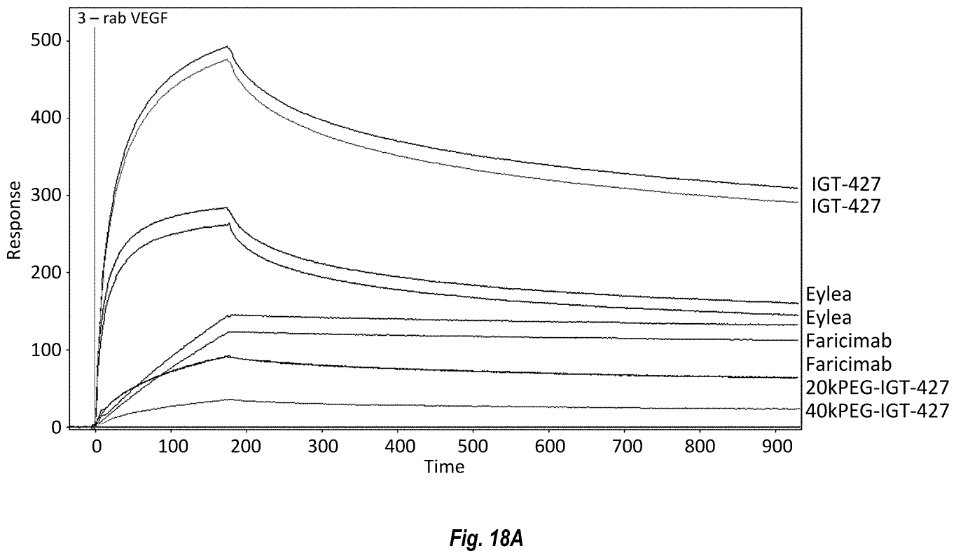

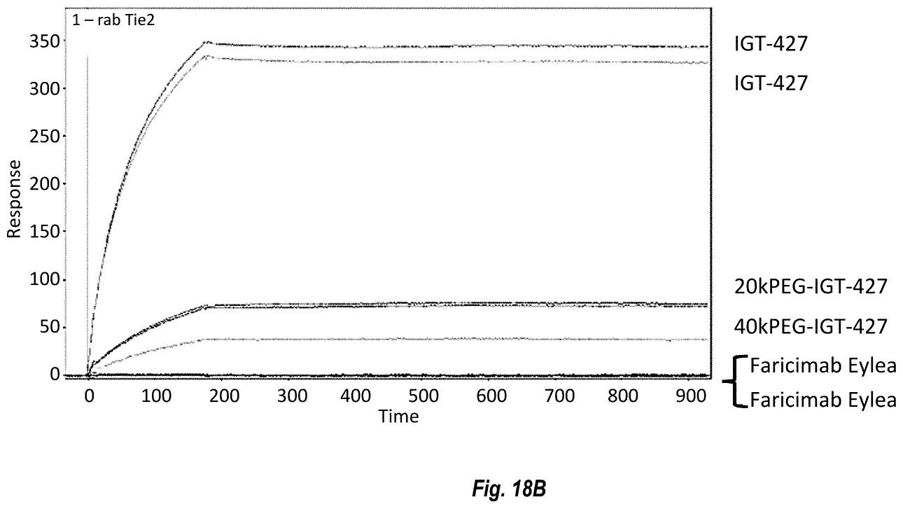

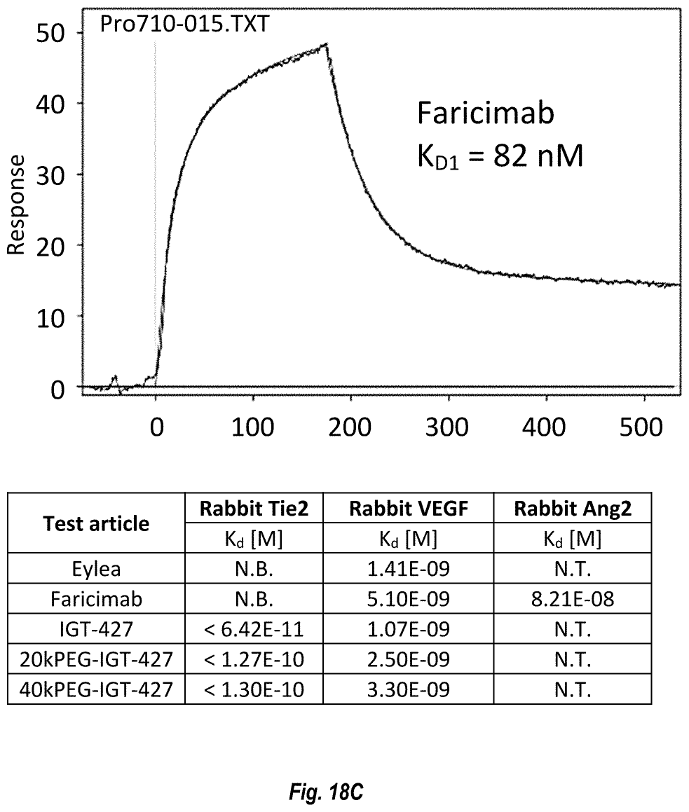

All test articles were formulated at 10 mg/mL and were greater than 90% purity by SEC-HPLC (FIG. 16) and non-reduced SDS-PAGE (FIG. 17), except for the 20 kDa PEGylated IGT-427, which contained 15% high molecular weight species. Endotoxin levels were all below 0.1 EU/mg and all test articles bound their respective antigens with the anticipated affinity (FIGS. 18A-18C).

### Example 20. Total Drug ELISAs for Vitreous Humor Measurements

Total drug levels in the vitreous humor were quantified by three different ELISAs, depending on the drug that was administered. IGT-427 and PEGylated versions of IGT-427 were captured onto a mouse anti-human IgG coated plate and detected with a Tie2-HRP conjugate. EYLEA® was captured onto a mouse anti-human IgG coated plate and detected with a polyclonal goat anti-human IgG-HRP conjugate. Finally, faricimab was captured onto a VEGF coated plate and detected with a polyclonal goat anti-human IgG-HRP conjugate. For all ELISA analyses, samples were assayed in duplicate between 1:1250 and 1:62500 dilution and the luminescence of the assay plate was read using a Molecular Devices SpectraMax® M5 plate reader. For all ELISA analyses, sample luminescence values were within the linear range of the standard curve. A 4-parameter equation was used to fit the entire standard curve and sample luminescence values were converted to concentrations via the 4-parameter fit.

Anti-human IgG capture/Tie2 detection ELISA. 1 μg/mL anti-human IgG in carbonate buffer (pH 9.5) was used to coat a high-binding plate at 4° C. overnight. The coated plate was washed with wash buffer (1× PBST (Phosphate Buffered Saline with TWEEN® 20) with 150 mM NaCl) and blocked for 1 hour at 37° C. with 5% BSA in PBST (shaking at 420 RPM). The blocked plate was washed, and samples, standard curve, and blanks were incubated on the plate for 1.5 hours at room temperature (shaking at 420 RPM). After sample incubation, the plate was washed and incubated with 1 μg/mL biotinylated human Tie2 for 1 hour at room temperature (covered, shaking at 420 RPM). The plate was washed again and was incubated with streptavidin-HRP for 1 hour protected from light. The plate was washed a final time and then developed with chemiluminescent HRP substrate. All luminescent wavelengths were read in a plate reader after 30 seconds.

Anti-human IgG capture/anti-huFc detection ELISA. 1 μg/mL anti-human IgG in carbonate buffer (pH 9.5) was used to coat a high-binding plate at 4° C. overnight. The coated plate was washed with wash buffer and blocked for 4 hours at 4° C. with 1% BSA in PBST. The blocked plate was washed and the plate was allowed to dry at 4° C. after the final wash. Samples, standard curve, and blanks were incubated on the plate overnight at 4° C. After sample incubation, the plate was washed and incubated with a polyclonal goat anti-human IgG-HRP conjugate for 1 hour at room temperature (covered, shaking at 420 RPM). The plate was washed a final time and then developed with chemiluminescent HRP substrate. All luminescent wavelengths were read in a plate reader after 30 seconds.

VEGF capture/anti-huFc detection ELISA. 1 μg/mL VEGF in carbonate buffer (pH 9.5) was used to coat a high-binding plate at 4° C. overnight. The coated plate was washed with wash buffer and blocked for 4 hours at 4° C. with 1% BSA in PBST. The blocked plate was washed and the plate was allowed to dry at 4° C. after the final wash. Samples, standard curve, and blanks were incubated on the plate overnight at 4° C. After sample incubation, the plate was washed and incubated with a polyclonal goat anti-human IgG-HRP conjugate for 1 hour at room temperature (covered, shaking at 420 RPM). The plate was washed a final time and then developed with chemiluminescent HRP substrate. All luminescent wavelengths were read in a plate reader after 30 seconds.

### Example 21. Measurement of Ocular PK

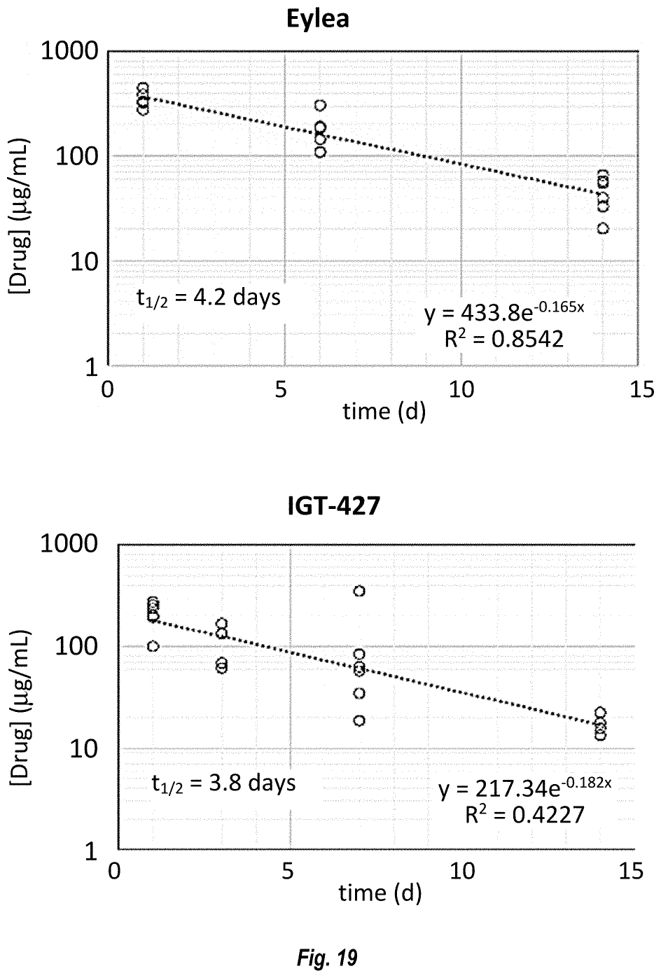

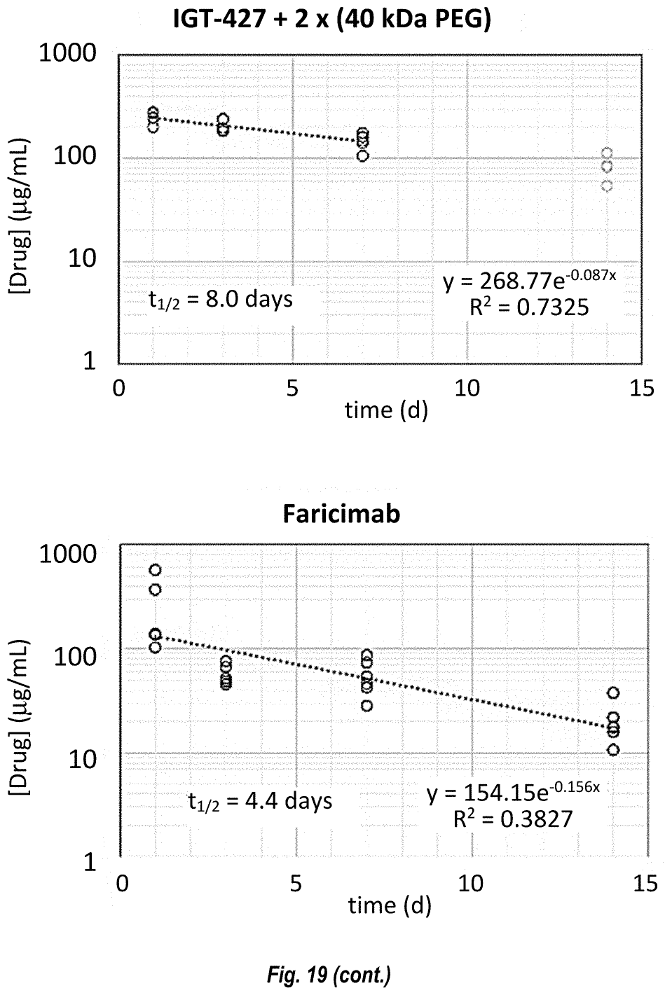

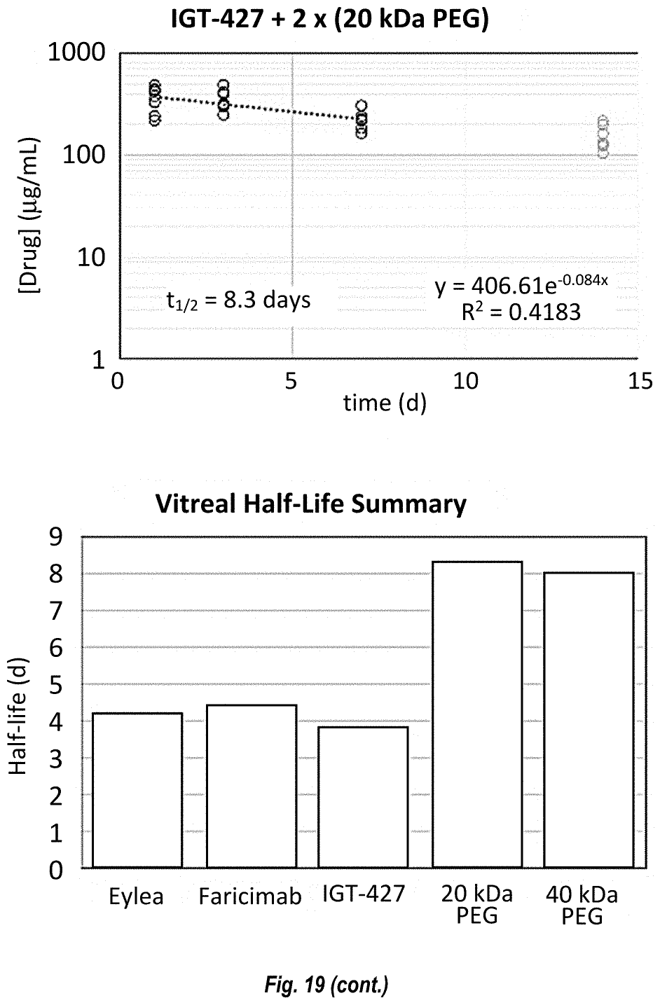

Total drug levels for each test article were measured in the vitreous humor at 1, 3, 7, and 14 days post-dose. At each time point, the eyes from three or four animals were harvested, resulting in either six or eight vitreous humor samples per test article per time point. Total drug levels from animals dosed with IGT-427 or PEGylated IGT-427 were quantified by an anti-human IgG capture and Tie2 detection ELISA. Total drug levels from animals dosed with EYLEA® were quantified by an anti-human IgG capture and anti-human Fc detection ELISA Finally, total drug levels from animals dosed with Faricimab were quantified by a VEGF capture and anti-human Fc detection ELISA. The results of each of these ELISAs are shown in FIG. 19. EYLEA® was measured in the vitreous with a Cmax value of 433.8 μg/mL and a half-life of 4.2 days. Faricimab was measured in the vitreous with a Cmax of 154.2 μg/mL and decays with a half-life of 4.4 days. IGT-427 was measured in the vitreous with a Cmax value of 217.3 μg/mL and a half-life of 3.8 days. IGT-427 that is modified with two 20 kDa linear PEG molecules was measured in the vitreous with a Cmax of 406.6 μg/mL and decays with a half-life of 8.3 days. IGT-427 that is modified with two 40 kDa branched PEG molecules was measured in the vitreous with a Cmax value of 268.8 μg/mL and a half-life of 8.0 days.

### INDUSTRIAL APPLICABILITY

The fusion protein that binds to Tie2 and VEGF can bind to Tie2 and VEGF with a high affinity, maintain cross-reactivity to humans, mice, and rabbits, and show the desired antigen reactivity. In addition, by inducing Tie2 phosphorylation, activation of the Tie2 receptor, and inhibition of VEGF, it can be used to prevent or treat angiogenic or vascular diseases of interest.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiments may be practiced in many ways and should be construed in accordance with the appended claims and any equivalents thereof.

When terms such as at least and about precede a list of numerical values or ranges, the terms modify all of the values or ranges provided in the list. In some instances, the term about may include numerical values that are rounded to the nearest significant figure. Any stated range includes the endpoints absent explicit language to the contrary; for example, “between 5 and 50” encompasses the values 5 and 50.

1. A fusion protein comprising an anti-Tie2 antibody or antigen-binding fragment thereof and a vascular endothelial growth factor (VEGF)-binding domain, wherein the fusion protein binds to Tie2 Ig3-FNIII (1-3) domain comprising the amino acid sequence of SEQ ID NO: 2, 3, or 4 and to VEGF.

2. The fusion protein of claim 1, wherein:
(a) the VEGF-binding domain comprises a VEGF receptor extracellular domain;
(b) the VEGF-binding domain comprises a VEGF-A binding region of VEGF receptor 1 (VEGFR1) of SEQ ID NO: 13 and a VEGF-A binding region of VEGF receptor 2 (VEGFR2) of SEQ ID NO: 14;
(c) the VEGF-binding domain is linked to the C-terminus of the heavy chain (HC) of the anti-Tie2 antibody or antigen-binding fragment thereof;
(d) the VEGF-binding domain comprises a sequence having at least 80%, 85%, 90%, 95%, 96%, 97%, 98%, or 99% identity to the amino acid sequence of SEQ ID NO: 15; and/or
(e) VEGF-binding domain comprises the amino acid sequence of SEQ ID NO: 15.

3. (canceled)

4. (canceled)

5. The fusion protein of claim 1, wherein:
(a) the fusion protein binds the amino acid sequence of SEQ ID NO: 20 of Tie2 and/or the amino acid sequence of SEQ ID NO: 21 of Tie2;
(b) the fusion protein binds to Tie2 Ig3-FNIII (1-3) domain comprising SEQ ID NO: 2, 3, or 4 with an affinity K_{D} (M) of less than 3E⁻⁹M; and/or
(c) the anti-Tie2 antibody or antigen-binding fragment thereof has an IgG1 isotype.

6. (canceled)

7. The fusion protein of claim 1, wherein the anti-Tie2 antibody or antigen-binding fragment thereof comprises a heavy chain variable region comprising heavy chain complementarity determining regions (CDRs) comprising amino acid sequences of SEQ ID NO:5-7 and a light chain variable region comprising a light chain CDRs comprising amino acid sequences of SEQ ID NO:8-10.

8. (canceled)

9. (canceled)

10. The fusion protein of claim 1, wherein the fusion protein comprises a linker between the VEGF-binding domain and the anti-Tie2 antibody or antibody fragment thereof.

11. The fusion protein of claim 10, wherein the linker comprises:
(a) a sequence having between 5 and 50 amino acid residues, between 10 and 40 residues, between 15 and 30 residues, or 20 residues;
(b) a sequence having at least 80%, 85%, 90%, 95%, 96%, 97%, 98%, or 99% identity to the amino acid sequence of SEQ ID NO: 16 or 25, or comprises the amino acid sequence of SEQ ID NO: 16 or 25; and/or
(c) amino acid sequence of SEQ ID NO: 16 or 25, and the VEGF-binding domain comprises the amino acid sequence of SEQ ID NO: 15.

12.-14. (canceled)

15. The fusion protein of claim 1, wherein the fusion protein comprises:
(a) a CH domain comprising a sequence having at least 80%, 85%, 90%, 95%, 96%, 97%, 98%, or 99% identity to the amino acid sequence of SEQ ID NO: 17, or comprising the amino acid sequence of SEQ ID NO: 17;
(d) one or more mutations in a heavy chain constant region that reduce or eliminate interaction with Fc Receptors;
(e) a heavy chain variable region comprising the amino acid sequence of SEQ ID NO:18 and a light chain variable region comprising the amino acid sequence of SEQ ID NO:19;
(f) a heavy chain comprising at least 80%, 85%, 90%, 95%, 96%, 97%, 98%, or 99% identity to the amino acid sequence of SEQ ID NO: 11 or 26, or the comprising the amino acid sequence of SEQ ID NO:11 or 26;
(g) a light chain comprising at least 80%, 85%, 90%, 95%, 96%, 97%, 98%, or 99% identity to the amino acid sequence of SEQ ID NO: 12, or comprising amino acid sequence of SEQ ID NO:12;
(h) the fusion protein is pegylated; and/or
(i) the fusion protein further comprises one or more half-life extension modulators.

16.-18. (canceled)

19. The fusion protein of claim 15, wherein the one or more mutations comprises a LALA mutation and mutations at K322 and P331 (EU numbering).

20. The fusion protein of claim 1, wherein the fusion protein comprises:
(a) one or more mutations at L₂₃₄, L₂₃₅, H310, M252, 1253, 5254, T256, H433, N434 and/or H435 (EU numbering); and/or
(b) C-terminal to the heavy chain constant domain or domains of the anti-Tie2 antibody or antigen-binding fragment thereof and in N- to C-terminal order, a linker between the VEGF binding domain and the anti-Tie2 antibody or antibody fragment thereof.

21. (canceled)

22. (canceled)

23. The fusion protein of claim 5, wherein the fusion protein comprises a CH domain comprising the amino acid sequence of SEQ ID NO: 17, a linker comprising amino acid sequence of SEQ ID NO: 16 or 25 between the VEGF-binding domain and the anti-Tie2 antibody or antibody fragment thereof, and a VEGF-binding domain comprising the amino acid sequence of SEQ ID NO: 15.

24.-28. (canceled)

29. The fusion protein of claim 15, wherein the fusion protein comprises a heavy chain comprising amino acid sequence of SEQ ID NO:11 or 26, and a light chain comprising amino acid sequence of SEQ ID NO:12.

30. (canceled)

31. (canceled)

32. The fusion protein of claim 15, wherein the fusion protein is site-specifically pegylated.

33. The fusion protein of claim 32, wherein the fusion protein is site-specifically pegylated on a cysteine residue.

34. The fusion protein of claim 15, wherein the fusion protein further comprises the sequence of SEQ ID NO: 22 and is site-specifically pegylated on the cysteine residue of the sequence of SEQ ID NO: 22.

35. The fusion protein of claim 34, wherein the sequence of SEQ ID NO: 22 is present at the C-terminus of the heavy chain or the heavy chain comprises the sequence of SEQ ID NO: 23 or 24.

36. (canceled)

37. (canceled)

38. The fusion protein of claim 5, wherein the polyethylene-glycol (PEG) has a molecular weight of about 40 kDa.

39. (canceled)

40. The fusion protein of claim 15, wherein the one or more half-life extension modulators comprises a chemical, biopolymer, or peptide that increases the half-life of the fusion protein.

41. The fusion protein of claim 40, wherein the one or more half-life extension modulators comprise: a biopolymer containing PEG (polyethylene-glycol), hyaluronic acid (HA), or phosphorylcholine; an albumin; an albumin-binding peptide, and/or an HA-binding protein fragment.

42. A nucleic acid encoding:
(a) the fusion protein of claim 1; or
(b) a set of one or more polynucleotides wherein each polynucleotide encodes at least one monomer chain of the fusion protein of claim 1, such that both light and heavy chains of said fusion protein are encoded.

43. An expression vector comprising the nucleic acid of claim 42.

44. (canceled)

45. A cell transformed with the expression vector of claim 43.

46. A method of manufacturing a fusion protein which binds Tie2 and VEGF, comprising the steps of:
culturing a cell of claim 45; and
recovering a fusion protein from the cultured cell.

47. A method for preventing or treating an angiogenic or vascular disease, or for regulating angiogenesis, endothelial signaling, inflammation, anoxia, and/or vascular leakage, comprising administering an effective amount of a fusion protein of claim 1 to a subject in need thereof.

48. (canceled)

49. (canceled)

50. The method of claim 47, wherein the angiogenic or vascular disease is cancer, metastasis, diabetic retinopathy, retinopathy of prematurity, diabetic macular edema, corneal graft rejection, macular degeneration, glaucoma optionally wherein the glaucoma is neovascular glaucoma, systemic erythrosis, proliferative retinopathy, psoriasis, hemophilic arthritis, allied sclerosis, capillary formation of atherosclerotic plaques, keloid, wound granulation, vascular adhesion, rheumatoid arthritis, osteoarthritis, autoimmune diseases, Crohn's disease, restenosis, atherosclerosis, intestinal adhesions, cat scratch disease, ulcer, liver cirrhosis, nephritis, diabetic nephropathy, diabetes mellitus, an inflammatory disease, or a neurodegenerative disease,
wherein the cancer is esophageal cancer, stomach cancer, large intestine cancer, rectal cancer, oral cancer, pharyngeal cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, or multiple myeloid blood cancer; and
wherein the inflammation is from sepsis, acute respiratory distress syndromes, and/or virus-infectious diseases.

51. (canceled)

52. (canceled)

53. (canceled)

54. The method of claim 47, wherein the subject is human.

55. The method claim 47, wherein the subject is a companion animal, such as wherein the companion animal is a dog, cat, rabbit, ferret, horse, mule, donkey, or hamster.

56. (canceled)

57. A pharmaceutical composition comprising:
(a) the fusion protein of claim 1;
(b) a nucleic acid molecule encoding a polypeptide comprising a chain monomer of the fusion protein of claim 1;
(c) a set of one or more polynucleotides, wherein each polynucleotide encodes at least one of the monomer chains of the fusion protein of claim 1, such that both light and heavy chains of said fusion protein are encoded; or
(d) a vector comprising a nucleic acid molecule encoding a polypeptide comprising a chain monomer of the fusion protein of claim 1,
and a pharmaceutical acceptable carrier, diluent or excipient.

58. A polypeptide comprising a heavy and/or light chain monomer of the fusion protein of claim 1.

59.-66. (canceled)

67. A nucleic acid molecule encoding a polypeptide of claim 58.

68.-83. (canceled)


SEQUENCE LISTING (30 sequences)

SEQ ID NO: 1 - protein, 1124 aa, Homo sapiens
  REGION 1..1124: note = Human Tie2 Full-Length
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 2 - protein, 390 aa, Homo sapiens
  REGION 1..390: note = Human Tie2 Ig3-FNIII(1-3)
TPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAP

SEQ ID NO: 3 - protein, 391 aa, Oryctolagus cuniculus
  REGION 1..391: note = Rabbit Tie2 Ig3-FNIII(1-3)
TPKIEDLPDHIEVNTGKFNPICKASGWPLPANEEMTLVKPDGTVLHPKDFNHTENFSVAIFTIHRILPLDSGVWVCSVNTVAGMVEKPFNISVKVLPKPLNAPNVIDTGHNFAVINISSEPYFGDGPIKSKKLLYKPVNDYEAWRHIQVTNEIVTLNYLEPRTEYELCVQLIRRGEGGEGHPGPVRRFTTASIGLPPPQGLILLPKSQTTLNLTWQPIFPSSEDDFYVEVERRSVQIKSDQQNIKVPGNLTSVLLNNLHPREQYVVRARVNTKAQGEWSEDLTAWTLSDIVPPQPENIKISNITDSSAVISWTILDGYSISSIIIRYKVQGKNEDQHIDVKIKNATITQYQLKGLEPETAYQVDMFAENNIGSSNPAFSHELMTLPESQAP

SEQ ID NO: 4 - protein, 389 aa, Mus musculus
  REGION 1..389: note = Mouse Tie2 Ig3-FNIII(1-3)
TPQIEDLPDHIEVNSGKFNPICKASGWPLPTSEEMTLVKPDGTVLQPNDFNYTDRFSVAIFTVNRVLPPDSGVWVCSVNTVAGMVEKPFNISVKVLPEPLHAPNVIDTGHNFAIINISSEPYFGDGPIKSKKLFYKPVNQAWKYIEVTNEIFTLNYLEPRTDYELCVQLARPGEGGEGHPGPVRRFTTASIGLPPPRGLSLLPKSQTALNLTWQPIFTNSEDEFYVEVERRSLQTTSDQQNIKVPGNLTSVLLSNLVPREQYTVRARVNTKAQGEWSEELRAWTLSDILPPQPENIKISNITDSTAMVSWTIVDGYSISSIIIRYKVQGKNEDQHIDVKIKNATVTQYQLKGLEPETTYHVDIFAENNIGSSNPAFSHELRTLPHSPAS

SEQ ID NO: 5 - protein, 5 aa, synthetic construct
  REGION 1..5: note = Antibody 3H7 CDRH1-KABAT
SYWMN

SEQ ID NO: 6 - protein, 17 aa, synthetic construct
  REGION 1..17: note = Antibody 3H7 CDRH2-KABAT
MIHPSDSETRLNQKFMD

SEQ ID NO: 7 - protein, 6 aa, synthetic construct
  REGION 1..6: note = Antibody 3H7 CDRH3-KABAT
GLYGNS

SEQ ID NO: 8 - protein, 11 aa, synthetic construct
  REGION 1..11: note = Antibody 3H7 CDRL1-KABAT
RASQDIGISLN

SEQ ID NO: 9 - protein, 7 aa, synthetic construct
  REGION 1..7: note = Antibody 3H7 CDRL2-KABAT
ATSSLDS

SEQ ID NO: 10 - protein, 9 aa, synthetic construct
  REGION 1..9: note = Antibody 3H7 CDRL3-KABAT
LQYASSPYT

SEQ ID NO: 11 - protein, 689 aa, synthetic construct
  REGION 1..689: note = IGT-427 HC with (G4S)4 linker
MNFGLRLIFLVLTLKGVQCEVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 12 - protein, 233 aa, synthetic construct
  REGION 1..233: note = IGT-427 LC
MNFGLRLIFLVLTLKGVQCDIQMTQSPSSLSASVGDRVTITCRASQDIGISLNWYQQEPGKAIKRLIYATSSLDSGVPKRFSGSRSGTEYTLTISSLESEDFADYYCLQYASSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 104 aa, synthetic construct
  REGION 1..104: note = VEGFR1
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 14 - protein, 101 aa, synthetic construct
  REGION 1..101: note = VEGFR2
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 15 - protein, 165 aa, synthetic construct
  REGION 1..165: note = Recombinant Human VEGF 165 Protein, VEGFR Binding Domain
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 16 - protein, 20 aa, synthetic construct
  REGION 1..20: note = (G4S)4 Linker
GGGGSGGGGSGGGGSGGGGS

SEQ ID NO: 17 - protein, 330 aa, synthetic construct
  REGION 1..330: note = IGT-427 CH Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 114 aa, synthetic construct
  REGION 1..114: note = IGT-427 VH (HC Variable Region)
EVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVS

SEQ ID NO: 19 - protein, 106 aa, synthetic construct
  REGION 1..106: note = IGT-427 VL (LC Variable Region)
DIQMTQSPSSLSASVGDRVTITCRASQDIGISLNWYQQEPGKAIKRLIYATSSLDSGVPKRFSGSRSGTEYTLTISSLESEDFADYYCLQYASSPYTFGGGTKLEI

SEQ ID NO: 20 - protein, 12 aa, synthetic construct
  REGION 1..12: note = Binding domain (amino acids 633-644 of Tie2 of SEQ ID NO: 1)
TLSDILPPQPEN

SEQ ID NO: 21 - protein, 14 aa, synthetic construct
  REGION 1..14: note = Binding domain (amino acids 713-726 of Tie2 of SEQ ID NO: 1)
FAENNIGSSNPAFS

SEQ ID NO: 22 - protein, 7 aa, synthetic construct
  REGION 1..7: note = C-terminal addition
GGSSKCA

SEQ ID NO: 23 - protein, 696 aa, synthetic construct
  REGION 1..696: note = IGT-427 HC with C-terminal addition
MNFGLRLIFLVLTLKGVQCEVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGSSKCA

SEQ ID NO: 24 - protein, 108 aa, synthetic construct
  REGION 1..108: note = VEGFR2 with C-terminal addition sequence from IGT-427 HC
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGSSKCA

SEQ ID NO: 25 - protein, 20 aa, synthetic construct
  REGION 1..20: note = IgG1 Linker
GGGGSGGGGSEPKSSDKTHT

SEQ ID NO: 26 - protein, 689 aa, synthetic construct
  REGION 1..689: note = IGT-427 HC with IgG1 linker
MGWSCIILFLVATATGVHSEVQLVQSGAEVKKPGASVKVSCKASGYSFTSYWMNWVRQAPGQGLEWMGMIHPSDSETRLNQKFMDRVTMTRDTSTSTVYMELSSLRSEDTAVYYCARGLYGNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 27 - DNA, 2067 bp, synthetic construct
  source 1..2067: mol_type = other DNA; note = Nucleotide sequence of IGT-427 HC with (G4S)4 linker
atgggatggtcatgtatcatcctttttctggtagcaactgcaactggagtacatagcgaggtgcagctggtgcaatcaggcgctgaggtcaagaaacccggggcttcagtcaaggtgagctgtaaggcttctggctactctttcacctcctactggatgaattgggtcagacaggcccctgggcaaggactggagtggatgggcatgatccacccaagcgacagtgaaacacgcctgaaccagaagtttatggaccgtgtcaccatgacaagagacaccagcacaagcactgtgtatatggagctctcttccctgcgctccgaagataccgccgtgtactactgcgcccggggactctatggaaactcctggggccagggcaccctagtgacagtgtcctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaagccgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctggcccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaggtggcggaggctctggaggcggcggatccggcggaggaggctccggaggaggtggcagcagtgatacgggaagacctttcgtggagatgtactctgagatccccgaaattattcacatgaccgagggccgggagctggtcatcccttgccgagtgacaagcccaaacatcactgttactctcaagaagtttcctctcgacaccctgataccggatggcaagagaatcatttgggattcccgcaagggcttcatcatctctaacgctacatataaggagatcggactgctgacatgcgaagccaccgtgaatggccacctctacaaaaccaactacctgacccaccgccaaacaaataccatcattgacgtggttctatccccaagccatgggattgaactgagtgtcggagaaaagttagtgctgaactgcaccgcacgcacggaactgaacgtcggcatcgactttaattgggagtacccctctagcaagcaccagcacaaaaagctggttaatcgagacctgaagacccagagcggttctgaaatgaagaaattcctgtccaccctgactattgacggggtaaccaggtcggatcaagggctgtatacttgcgcggcgtctagtggcttgatgacaaagaagaacagcaccttcgttagagtgcatgagaaa

SEQ ID NO: 28 - DNA, 702 bp, synthetic construct
  source 1..702: mol_type = other DNA; note = Nucleotide sequence of IGT-427 LC
atggagacagacacactcctgctatgggtactgctgctctgggttccaggctccaccggcgacattcagatgacccaatcgccgtcttccctctctgctagcgtcggcgaccgagttaccatcacttgtcgagctagccaggacatcggtatctcactgaattggtaccagcaagagccaggaaaagccatcaagaggctgatatatgccacaagttcactggatagcggagtgcccaaaaggtttagtggcagtagaagcggcacagagtataccctgactatctcaagcctggagtccgaggactttgctgactactattgcctacaatacgcgtcaagtccctacacatttggcgggggaacaaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 29 - DNA, 2067 bp, synthetic construct
  source 1..2067: mol_type = other DNA; note = Nucleotide sequence of IGT-427 HC with IgG1 linker
atgggatggtcatgtatcatcctttttctggtagcaactgcaactggagtacatagcgaggtccagttagtgcaatcgggagccgaagtcaagaagccaggtgcctccgttaaagtttcatgtaaagcttcaggatacagtttcacatcttattggatgaattgggttagacaggctccaggtcaaggcctggagtggatgggcatgatccatccttctgatagcgaaacccgcctgaaccagaagttcatggacagagttaccatgacccgggacaccagcactagcaccgtatacatggagctgagtagcctgaggtcagaggatacagccgtgtattactgtgcacgtggcctttatggcaattcttggggccagggcactttggtaacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaagccgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctggcccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaaggaggaggcggctctggtggtggaggcagtgaacccaagagcagcgacaagacacatacaagtgacacaggccggccattcgtggaaatgtattccgaaatacctgagatcattcacatgacagagggcagagagctggtcattccgtgtcgggtcacatcccctaatatcactgtgaccttgaaaaagttccccctggataccctgattcctgacggaaagcgcatcatctgggacagcaggaaagggttcatcatctctaacgccacctacaaggaaattggcttgctgacatgtgaagcaaccgtcaacgggcacctgtacaagaccaactatctgactcatcggcaaactaataccatcatcgatgtggtgctctctccaagtcatggaattgagctctccgttggcgagaaactggtgctcaattgcaccgcacggacggagttgaacgtcggaatcgattttaattgggagtacccctcctcgaagcatcagcacaagaagctcgtaaaccgagatctcaaaacccagtccggctctgagatgaagaaattcctgagcacactgacaattgatggagtgacccgaagcgaccagggcctctacacctgcgctgctagctccggattgatgaccaagaagaactccactttcgtccgagtgcacgagaag

SEQ ID NO: 30 - protein, 69 aa, synthetic construct
  REGION 1..69: note = IGT-427 CL1 Domain
HTTPSSANTACRUZBREAKERSSMUGMUGCOMSANTACRUZBREAKERSPHOTOSHOOTGIRLSGREEN